(19) United States
(12) Patent Application Publication
(10) Pub. No.: US 2010/0008917 A1
(43) Pub. Date: Jan. 14, 2010

(54) TREATMENT OF APLASTIC ANEMIA

(76) Inventors: Nancy Hosken, Seattle, WA (US); Kelly Byrnes-Blake, Duvall, WA (US); Ty Brender, Seattle, WA (US); Monica J. Huber, Mill Creek, WA (US); Margaret D. Moore, Seattle, WA (US); Shirley Rene, Seattle, WA (US); Mark W. Rixon, Issaquah, WA (US); Sara Underwood, Snohomish, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 12/438,909

(22) PCT Filed: Aug. 27, 2007
(86) PCT No.: PCT/US07/76899

§ 371 Date: Aug. 5, 2009

Related U.S. Application Data
(60) Provisional application No. 60/823,597, filed on Aug. 25, 2006.

(51) Int. Cl. A61K 39/395

(52) U.S. Cl. 424/134.1; 424/133.1; 424/135.1

(57) ABSTRACT

Methods of treating aplastic anemia in a patient and of increasing blood cell production in a patient having aplastic anemia are disclosed. The methods comprise administering to the patient a therapeutically effective amount of an IL-27 antagonist in combination with a pharmaceutically acceptable vehicle. IL-27 antagonists include soluble IL-27RA proteins and antagonists that comprise an antigen-binding site of an antibody.

### BACKGROUND OF THE INVENTION

Aplastic anemia is a rare disorder characterized by a slowing or cessation of blood cell production caused by destruction of stem cells in the bone marrow. This destruction is believed to result from an aberrant immune response wherein hematopoietic cells are destroyed by T lymphocytes. The resulting deficit in red cells, white cells, and platelets leads to fatigue and increased risk of infection and uncontrolled bleeding. Untreated, it can be fatal. The condition affects an estimated two people per million per year in the United States, with an incidence at least two to three times higher in Asian countries. Both acquired and hereditary forms of aplastic anemia occur.

Acquired aplastic anemia is the more common type. Causes include exposure to toxic chemicals (including inhaled solvents), chemotherapy and other drugs, radiation, and viruses (e.g., hepatitis, Epstein-Barr virus, cytomegalovirus, parvovirus B19, and HIV), although in many cases no cause is identified. Autoimmune diseases, diseases of the bone marrow, and, rarely, pregnancy are also associated with aplastic anemia.

Hereditary aplastic anemia is rare, occurring with inherited conditions such as Fanconi anemia, Shwachman-Diamond syndrome, and dyskeratosis congenita.

Current treatments for aplastic anemia include transfusion of blood or blood components (red cells or platelets), antibiotics/anti-infective drugs, immune-suppressing drugs, bone marrow transplantation, colony stimulating factors, and erythropoietin.

### SUMMARY OF THE INVENTION

Within one aspect of the invention there is provided a method of treating aplastic anemia in a patient, comprising administering to a patient having aplastic anemia a therapeutically effective amount of an IL-27 antagonist in combination with a pharmaceutically acceptable vehicle.

Within a second aspect of the invention there is provided a method of increasing blood cell production in a patient having aplastic anemia, comprising administering to the patient a therapeutically effective amount of an IL-27 antagonist in combination with a pharmaceutically acceptable vehicle.

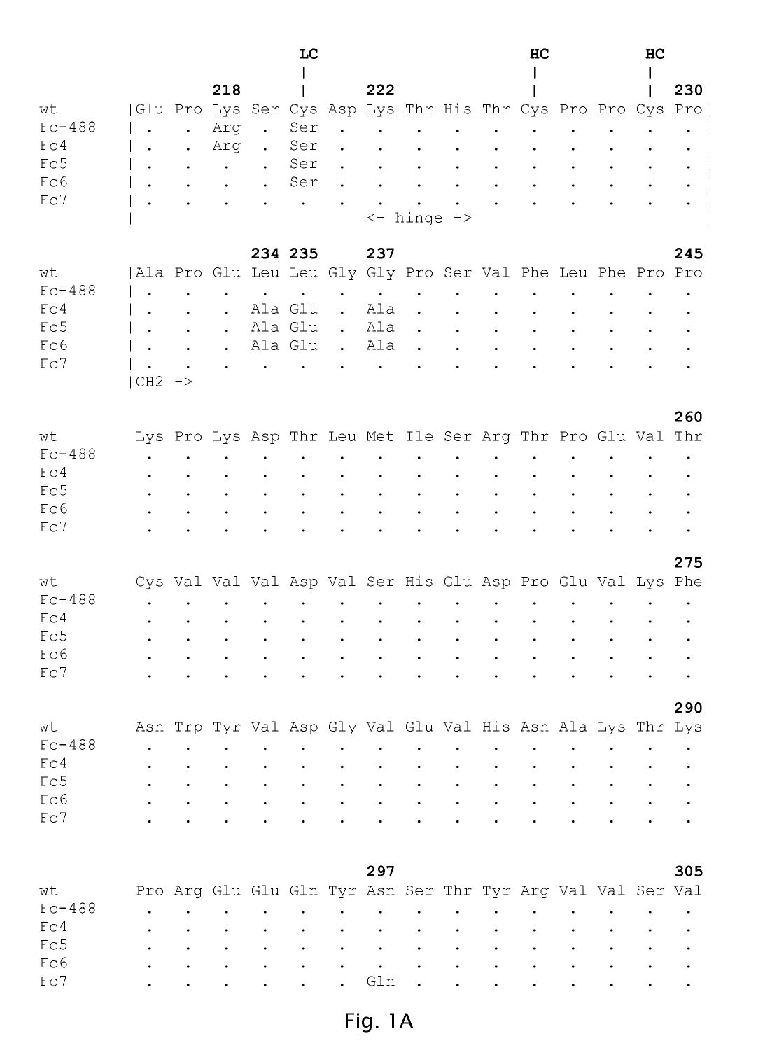

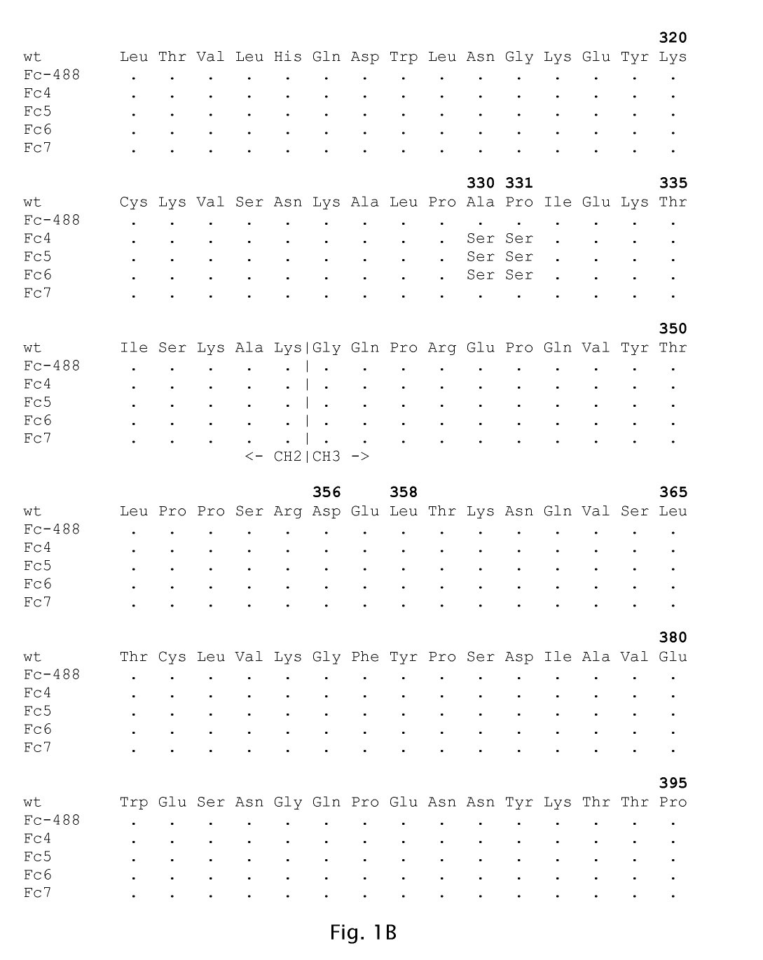

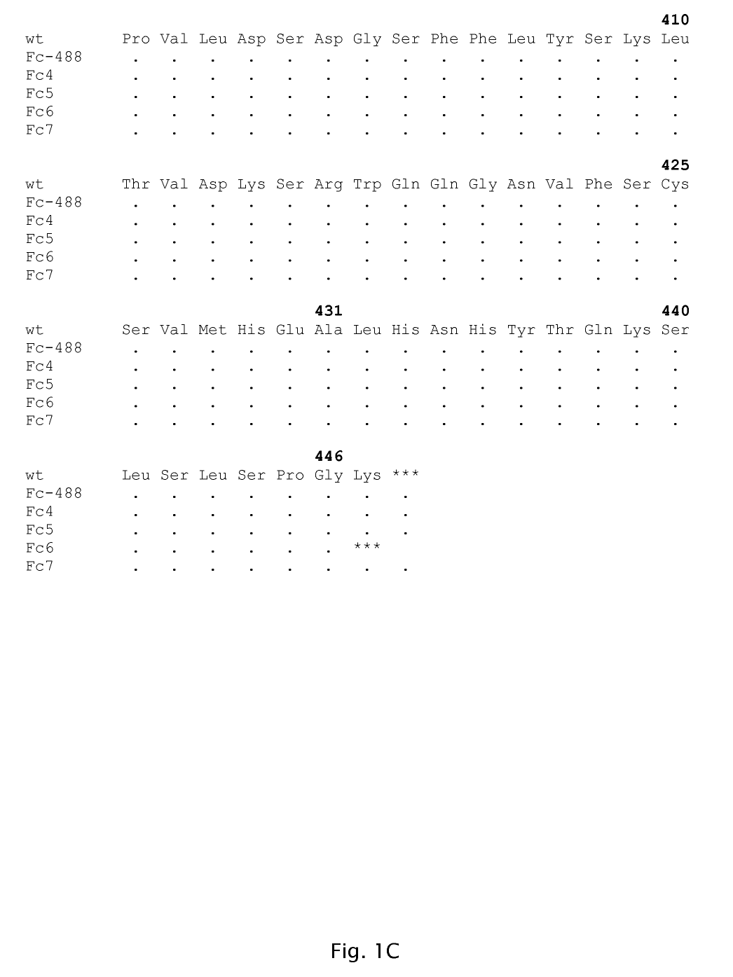

Within certain embodiments of the invention, the antagonist is a soluble IL-27RA protein that binds to and reduces the activity of IL27. Within one embodiment, the soluble IL-27RA protein is a disulfide linked dimer, wherein each chain of the dimer comprises an extracellular ligand-binding domain of an IL-27RA joined to an immunoglobulin fragment comprising a heavy chain CH3 domain (or “IL27RA-Fc fusion” or “immunoglobulin-IL-27RA fusion”). Within a related embodiment, each chain of the dimer further comprises an immunoglobulin hinge between the extracellular ligand binding domain and the CH3 domain. Within another related embodiment, the immunoglobulin fragment is an immunoglobulin Fc fragment. Fc fragments within this embodiment include wild-type Fc fragments; Fc fragments containing an amino acid substitution that reduces binding of the Fc fragment to Fc.gamma.RI, reduces complement fixation, or replaces a cysteine residue that normally forms a disulfide bond with an immunoglobulin light chain; and Fc fragments consisting of a sequence of amino acid residues selected from the group consisting of the sequences shown in FIGS. 1A-1C. Within a further embodiment of the invention, the soluble IL-27RA protein is a dimer. Within another embodiment, the soluble IL-27RA protein comprises amino acid residues 33 to 744 of SEQ ID NO:3.

Within other embodiments of the invention, the antagonist comprises an antigen-binding site of an antibody and the antagonist specifically binds to IL27RA, EBI3, IL-27 p28, or an EBI3/IL-27 p28 heterodimer. Within related embodiments, the antagonist is an antibody, such as a monoclonal antibody. The monoclonal antibody may be a humanized monoclonal antibody. Within another embodiment, the antagonist is a monoclonal antibody that specifically binds to IL27RA. Within a further embodiment, the antagonist is an Fv fragment, single-chain Fv fragment, Fab fragment, Fab′ fragment, F(ab′)₂ fragment, diabody, minibody, or Fab-scFv fusion.

Within an additional embodiment of the invention, the aplastic anemia is acquired aplastic anemia.

Within further embodiments of the invention, the IL-27 antagonist is administered in combination with an IL-12 antagonist. IL-12 antagonists for use within these embodiments include, for example, anti-IL-12 antibodies, anti-IL-12 receptor antibodies, and soluble IL-12 receptors.

These and other aspects of the invention will become evident upon reference to the following detailed description of the invention and the attached drawings.

### BRIEF DESCRIPTION OF THE FIGURES

The drawing (FIGS. 1A-1C) illustrates the amino acid sequences of certain immunoglobulin Fc polypeptides (SEQ ID NO: 1). Amino acid sequence numbers are based on the EU index (Kabat et al., Sequences of Proteins of Immunological Interest, U.S. Department of Health and Human Services, NIH, Bethesda, 1991). The illustrated sequences include a wild-type human sequence (“wt”) and five variant sequences, designated Fc-488, Fc4, Fc5, Fc6, and Fc7. The Cys residues normally involved in disulfide bonding to the light chain constant region (LC) and heavy chain constant region (HC) are indicated. A “.” indicates identity to wild-type at that position. *** indicates the stop codon; the C-terminal Lys residue has been removed from Fc6. Boundaries of the hinge, C_{H}2, and C_{H}3 domains are shown.

### DESCRIPTION OF THE INVENTION

As used herein, the term “antagonist” denotes a compound that reduces the activity of another compound in a biological setting. Thus, an IL-27 antagonist is a compound that reduces the activity of IL-27. Antagonists include, without limitation, antibodies and soluble receptors that bind to a ligand (e.g., IL-27) or its receptor, thereby interfering with ligand-receptor interactions and/or other receptor functions.

The term “antibody” is used herein to denote proteins produced by the body in response to the presence of an antigen and that bind to the antigen, as well as antigen-binding fragments and engineered variants thereof. Hence, the terms “antibody” and “antibodies” include polyclonal antibodies, affinity-purified polyclonal antibodies, monoclonal antibodies, and antigen-binding antibody fragments, such as F(ab′)₂ and Fab fragments. Genetically engineered intact antibodies and fragments, such as chimeric antibodies, humanized antibodies, single-chain Fv fragments, single-chain antibodies, diabodies, minibodies, linear antibodies, multivalent or multispecific hybrid antibodies, and the like are also included. Thus, the term “antibody” is used expansively to include any protein that comprises an antigen binding site of an antibody and is capable of binding to its antigen.

Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, human cellular and humoral effector mechanisms can be fully exploited, and the potential for adverse immune reactions upon administration to humans is reduced.

An “antigen-binding site of an antibody” is that portion of an antibody that is sufficient to bind to its antigen. The minimum such region is a variable domain. Single-domain binding sites can be generated from camelid antibodies (Muyldermans and Lauwereys, J. Mol. Recog. 12(2):131-140, 1999; Nguyen et al., EMBO J. 19:921-930, 2000) or from VH domains of other species to produce single-domain antibodies (“dAbs”; see, Ward et al., Nature 341:544-546, 1989; Winter et al., U.S. Pat. No. 6,248,516). More commonly, an antigen-binding site of an antibody comprises both a heavy chain variable domain and a light chain variable domain that bind to a common epitope. Within the present invention, a molecule that “comprises an antigen-binding site of an antibody” may further comprise one or more of a second antigen-binding site of an antibody (which may bind to the same or a different epitope or to the same or a different antigen), a peptide linker, an immunoglobulin constant domain, an immunoglobulin hinge, an amphipathic helix (Pack and Pluckthun, Biochem. 31:1579-1584, 1992), a non-peptide linker, an oligonucleotide (Chaudri et al., FEBS Letters 450:23-26, 1999), and the like, and may be a monomeric or multimeric protein. Examples of molecules comprising an antigen-binding site of an antibody are known in the art and include, for example, Fv fragments, single-chain Fv fragments (scFv), Fab fragments, diabodies, minibodies, Fab-scFv fusions, bispecific (scFv)₄-IgG, and bispecific (scFv)₂-Fab. See, for example, Hu et al., Cancer Res. 56:3055-3061, 1996; Atwell et al., Molecular Immunology 33:1301-1312, 1996; Carter and Merchant, Curr. Opin. Biotechnol. 8:449-454, 1997; Zuo et al., Protein Engineering 13:361-367, 2000; and Lu et al., J. Immunol. Methods 267:213-226, 2002.

“Chimeric antibodies” are antibodies whose light and heavy chain genes have been constructed, typically by genetic engineering, from immunoglobulin variable and constant region genes belonging to different species. For example, the variable segments of the genes from a mouse monoclonal antibody may be joined to human constant region-encoding segments (e.g., human gamma 1 or gamma 3 heavy chain genes, and human kappa light chain genes). A therapeutic chimeric antibody is thus a hybrid protein, typically composed of the variable or antigen-binding domains from a mouse antibody and the constant domains from a human antibody, although other mammalian species may be used.

An “immunoglobulin” is a serum protein that functions as an antibody in a vertebrate organism. Five classes of immunoglobulin protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol .gamma. For example, immunoglobulins of the IgG1 subclass contain a .gamma.1 heavy chain constant region. Each immunoglobulin heavy chain possesses a constant region that consists of constant region protein domains (C_{H}1, hinge, C_{H}2, and C_{H}3; IgG3 also contains a C_{H}4 domain) that are essentially invariant for a given subclass in a species. DNA sequences encoding human and non-human immunoglobulin chains are known in the art. See, for example, Ellison et al., DNA 1:11-18, 1981; Ellison et al., Nucleic Acids Res. 10:4071-4079, 1982; Kenten et al., Proc. Natl. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228. For a review of immunoglobulin structure and function see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 Kd or 214 amino acids) are encoded by a variable region gene at the NH₂-terminus (encoding about 110 amino acids) and a by a kappa or lambda constant region gene at the COOH-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y., 1989), Ch. 7).

As used herein, the terms “single-chain Fv” and “single-chain antibody” refer to antibody fragments that comprise, within a single polypeptide chain, the variable regions from both heavy and light chains, but lack constant regions. In general, a single-chain antibody further comprises a polypeptide linker between the V_{H} and V_{L} domains, which enables it to form the desired structure that allows for antigen binding. Single-chain antibodies are discussed in detail by Pluckthun in The Pharmacology of Monoclonal Antibodies, vol. 113, Rosenburg and Moore eds., Springer-Verlag, N.Y., pp. 269-315 (1994). See also, WIPO Publication WO 88/01649; U.S. Pat. Nos. 4,946,778 and 5,260,203; and Bird et al., Science 242:423-426, 1988. Single-chain antibodies can also be bi-specific and/or humanized.

A “Fab fragment” contains one light chain and the C_{H}1 and variable regions of one heavy chain. The heavy chain of a Fab fragment cannot form a disulfide bond with another heavy chain molecule.

A “Fab′ fragment” contains one light chain and one heavy chain that contains more of the constant region, between the C_{H}1 and C_{H}2 domains, such that an interchain disulfide bond can be formed between two heavy chains to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between two heavy chains.

An immunoglobulin “Fc fragment” (or Fc domain) is the portion of an antibody that is responsible for binding to antibody receptors on cells and the C1q component of complement. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, C_{H}2, and C_{H}3 domains. However, more recently the term has been applied to a single chain consisting of C_{H}3, C_{H}2, and at least a portion of the hinge sufficient to form a disulfide-linked dimer with a second such chain. For a review of immunoglobulin structure and function see Putnam, The Plasma Proteins, Vol V, Academic Press, Inc., 49-140, 1987; and Padlan, Mol. Immunol. 31:169-217, 1994. As used herein, the term Fc includes variants of naturally occuring sequences.

An immunoglobulin “Fv” fragment contains a heavy chain variable domain (V_{H}) and a light chain variable domain (V_{L}), which are held together by non-covalent interactions. An immunoglobulin Fv fragment thus contains a single antigen-binding site. The dimeric structure of an Fv fragment can be further stabilized by the introduction of a disulfide bond via mutagenesis. See, Almog et al., Proteins 31:128-138, 1998.

A “polypeptide” is a polymer of amino acid residues joined by peptide bonds, whether produced naturally or synthetically. Polypeptides of less than about 10 amino acid residues are commonly referred to as “peptides”.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A “soluble receptor” is a receptor polypeptide that is not bound to a cell membrane. Soluble receptors are most commonly ligand-binding receptor polypeptides that lack transmembrane and cytoplasmic domains. Soluble receptors can comprise additional amino acid residues, such as affinity tags that provide for purification of the polypeptide or provide sites for attachment of the polypeptide to a substrate, or immunoglobulin constant region sequences. See, for example, Nilsson et al., EMBO J. 4:1075, 1985; Nilsson et al., Methods Enzymol. 198:3, 1991; Smith and Johnson, Gene 67:31, 1988; Grussenmeyer et al., Proc. Natl. Acad. Sci. USA 82:7952-7954, 1985; Hopp et al., Biotechnology 6:1204-1210, 1988; Kellerman and Ferenci, Methods Enzymol. 90:459-463, 1982; Guan et al., Gene 67:21-30, 1987; and Ford et al., Protein Expression and Purification 2: 95-107, 1991. DNAs encoding affinity tags and other reagents are available from commercial suppliers (e.g., STRATAGENE, La Jolla, Calif.; Sigma-Aldrich, St. Louis, Mo.; New England Biolabs, Beverly, Mass.). Two copies of a soluble receptor may be joined using a flexible linker (typically a glycine-rich polypeptide) as disclosed by, for example, Fischer et al., Nature Biotech. 15:142, 1997 and U.S. Pat. No. 5,073,627. Many cell-surface receptors have naturally occurring, soluble counterparts that are produced by proteolysis. Receptor polypeptides are said to be substantially free of transmembrane and intracellular polypeptide segments when they lack sufficient portions of these segments to provide membrane anchoring or signal transduction, respectively.

All references cited herein are incorporated by reference in their entirety.

The present invention provides methods of treating aplastic anemia by the administration to a patient of an IL-27 antagonist. IL-27 antagonists include molecules that bind to IL-27 or its receptor and thereby reduce the activity of IL-27 on cells that express the receptor. In particular, IL-27 antagonists include soluble forms of IL-27RA and antibodies that specifically bind to IL-27RA, EBI3, IL-27 p28, or an EBI3/IL-27 p28 heterodimer. In addition, binding proteins based on non-antibody scaffolds (see, e.g., Koide et al., J. Mol. Biol. 284:1141-1151, 1998; Hosse et al. Protein Sci. 15:14-27, 2006 and references therein) may be employed. A representative human IL-27RA protein is shown in SEQ ID NO:5 This protein has been disclosed in U.S. Pat. No. 5,792,850, wherein it is referred to as “Zcytor1.” Preferred IL-27 antagonists for use within the invention include soluble receptors (including fusion proteins comprising the cytokine-binding domain of an IL-27RA (or “Zcytor1 fragment”) fused to an immunoglobulin Fc fragment) and antibodies that specifically bind to IL-27RA.

The Zcytor1 fragment preferably has at least 80% amino acid sequence identity with the amino acid structure of the extracellular domain of SEQ ID NO: 5, though said fragment may have at least 80% amino acid sequence identity with amino acid residue 1 to amino acid residue 578 of SEQ ID NO:5. Thus, said Zcytor1 fragment may comprise one or more of the extracellular domain, the transmembrane domain, the intracellular signaling domain, the cytokine binding domain, a fibronectin domain, a plurality of fibronectin domains and a plurality of cytokine binding domains. In one embodiment, said Zcytor1 fragment has an amino acid sequence that is at least 80% identical to residue 1 to about residue 514 of SEQ ID NO:5. In another embodiment, said Zcytor1 fragment has an amino acid sequence that is at least 80% identical to residues 33 to 514 of SEQ ID NO:5. In another embodiment, said Zcytor1 fragment has an amino acid sequence that is at least 80% identical to residues 33 to 235 of SEQ ID NO:5. In a still further embodiment, said Zcytor1 fragment comprises one or more of said conserved residues, with reference to SEQ ID NO:5: a Cys-X-Trp domain at residues 52-54, a Cys residue at position 41, a Trp residue at position 151, and an Arg residue at position 207. An alternatively spliced form of human IL-27RA having a additional 58 amino acids in the cytoplasmic domain is shown in SEQ ID NO:34, which may also be used as the Zcytor1 fragment of the IL27RA-Fc fusion protein, as described above.

As is used herein, the term “at least 80% identity” means that an amino acid sequence shares 80%-100% identify with a reference sequence. This range of identity is inclusive of all whole (e.g., 85%, 87%, 93%, 98%) or partial numbers (e.g., 87.27%, 92.83%, 98.11%—to two significant figures) embraced within the recited range numbers, therefore forming a part of this description. For example, an amino acid sequence with 200 residues that share 85% identity with a reference sequence would have 170 identical residues and 30 non-identical residues. Similarly, the amino acid sequence may have 200 residues that are identical to a reference sequence that is 235 residues in length, thus the amino acid sequence will be 85.11% identical to the larger reference sequence. This scenario is more typical when an amino acid sequence is a portion of a domain on the reference sequence. Amino acid sequences may additionally vary in percent identity from a reference sequence by way of both size differences and residue mismatches. Those ordinarily skilled in the are will readily calculate percent identity between an amino acid and a reference sequence.

As noted above, IL-27 is a heterodimer of EBI3 and IL27 p28 (Pflanz et al., ibid.). EBI3 is a secreted, 34 kDa glycoprotein that is related to the IL-12 p40 subunit. EBI3 DNA and protein sequences are disclosed by Birkenbach et al., U.S. Pat. No. 6,043,351; Devergne et al., J. Virol. 70:1143-1153, 1996; and Timans et al., U.S. Patent Application Publication No. 2004/0198955 A1. Human and mouse IL-27 p28 sequences are disclosed by Pflanz et al. (ibid.) and Timans et al. (ibid.).

Methods for preparing antibodies are disclosed below. This disclosure uses IL-27RA as an exemplary antigen (antibody target). Those skilled in the art will recognize that this disclosure is also applicable to other antigens, including EBI3, IL-27 p28, and EBI3/IL-27 p28 heterodimers.

Methods for preparing and isolating polyclonal antibodies, monoclonal antibodies, and antigen-binding antibody fragments thereof are well known in the art. See, for example, Cooligan, et al. (eds.), Current Protocols in Immunology, John Wiley and Sons, Inc., 2006; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982. Antigen binding fragments, including scFv, can be prepared using phage display libraries according to methods known in the art. Phage display can also be employed for the preparation of binding proteins based on non-antibody scaffolds (Koide et al., ibid.). Methods for preparing recombinant human polyclonal antibodies are disclosed by Wiberg et al., Biotechnol Bioeng. 94(2):396-405, 2006; Meijer et al., J. Mol. Biol. 358(3):764-772, 2006; Haurum et al., U.S. 20020009453 A1; and Haurum et al., U.S. 20050180967 A1.

As would be evident to one of ordinary skill in the art, polyclonal antibodies for use within the present invention can be generated by inoculating any of a variety of warm-blooded animals such as horses, cows, goats, sheep, dogs, chickens, rabbits, mice, and rats with an IL-27RA polypeptide or a fragment thereof. The immunogenicity of an IL-27RA polypeptide can be increased through the use of an adjuvant, such as alum (aluminum hydroxide) or Freund's complete or incomplete adjuvant. Polypeptides useful for immunization also include fusion polypeptides, such as fusions of IL-27RA or a portion thereof with an immunoglobulin polypeptide or with maltose binding protein. The polypeptide immunogen may be a full-length molecule or a portion thereof. If the polypeptide portion is hapten-like, it may be advantageously joined or linked to a macromolecular carrier (such as keyhole limpet hemocyanin (KLH), bovine serum albumin (BSA) or tetanus toxoid) for immunization.

Antibodies are considered to be specifically binding if 1) they exhibit a threshold level of binding activity, and 2) they do not significantly cross-react with control polypeptide molecules. A threshold level of binding is determined if an anti-IL-27RA antibody binds to an IL-27RA polypeptide, peptide or epitope with an affinity at least 10-fold greater than the binding affinity to a control (non-IL-27RA) polypeptide. It is preferred that antibodies used within the invention exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, commonly by surface plasmon resonance using automated equipment. Other methods are known in the art, for example Scatchard analysis (Scatchard, Ann. NY Acad. Sci. 51:660-672, 1949).

In addition, antibodies can be screened against known IL-27RA-related polypeptides (e.g., orthologs, paralogs, or sequence variants) to isolate a population of antibodies that is highly specific for binding to a particular IL-27RA protein or polypeptide. Such highly specific populations include, for example, antibodies that bind to human IL-27RA but not to mouse IL-27RA. Such a lack of cross-reactivity with related polypeptide molecules is shown, for example, by the antibody detecting an IL-27RA polypeptide but not known, related polypeptides using a standard Western blot analysis (Ausubel et al., eds., Current Protocols in Molecular Biology, Green and Wiley and Sons, N.Y., 1993) or ELISA (enzyme immunoassay) (Chan D. W. ed., Immunoassay, A Practical Guide, Academic Press, Inc. 1987). In another example, antibodies raised to an IL-27RA polypeptide are adsorbed to related polypeptides adhered to insoluble matrix; antibodies that are highly specific to the IL-27RA polypeptide will flow through the matrix under the proper buffer conditions. Screening allows isolation of polyclonal and monoclonal antibodies non-crossreactive to known, closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2:67-101, 1984.

For use within the present invention, monoclonal antibodies (“mAbs”) can be prepared by immunizing subject animals, for example rats or mice, with a purified IL-27RA protein or fragment thereof. In a typical procedure, rats are each given an initial intraperitoneal (IP) injection of the purified protein or fragment, typically in combination with an adjuvant (e.g., Complete Freund's Adjuvant or RIBI Adjuvant (available from Sigma-Aldrich, St. Louis, Mo.)) followed by booster IP injections of the purified protein at, for example, two-week intervals. Seven to ten days after the administration of the third booster injection, the animals are bled and the serum is collected. Additional boosts can be given as necessary.

Splenocytes and lymphatic node cells are harvested from high-titer animals and fused to myeloma cells (e.g., mouse SP2/0 or Ag8 cells) using conventional methods. The fusion mixture is then cultured on a feeder layer of thymocytes or cultured with appropriate medium supplements (including commercially available supplements such as Hybridoma Fusion and Cloning Supplement; Roche Diagnostics, Indianapolis, Ind.). About 10 days post-fusion, specific antibody-producing hybridoma pools are identified using standard assays (e.g., ELISA). Positive pools may be analyzed further for their ability to block or reduce the activity of the target protein. Positive pools are cloned by limiting dilution.

The invention also includes the use of multiple monoclonal antibodies that are specific for different epitopes on a single target molecule. Use of such multiple antibodies in combination can reduce carrier effects seen with single antibodies and may also increase rates of clearance via the Fc receptor and improve ADCC. Two, three, or more monoclonal antibodies can be used in combination.

The amino acid sequence of a native antibody can be varied through the application of recombinant DNA techniques. Thus, antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Examples of engineered constant region sequences are shown in FIGS. 1A-1C (SEQ ID NO: 1). Changes in the variable region will be made in order to improve the antigen binding characteristics. Phage display techniques can also be employed. See, for example, Huse et al., Science 246:1275-1281, 1989 and Ladner et al., U.S. Pat. No. 5,571,698.

For large-scale production, antibody-encoding genes are cloned and expressed in cultured mammalian cells, commonly Chinese hamster ovary (CHO) cells, although other cell lines known in the art can be employed. Variable region genes for an antibody of interest can be cloned by PCR using degenerate V region primers. The cloned V region genes are joined to the desired constant region genes to produce complete antibody coding sequences, which are then screened to verify that the encoded antibody has the desired binding specificity. For therapeutic antibodies for use in humans it is usually desirable to humanize the non-human regions of an antibody according to known procedures. See, for example, U.S. Pat. Nos. 5,530,101; 5,821,337; 5,585,089; 5,693,762; and 6,180,370. However, non-humanized chimeric antibodies can be used therapeutically in immunosuppressed patients.

Human antibodies can also be made in transgenic, non-human animals, commonly mice. See, e.g., Tomizuka et al., U.S. Pat. No. 7,041,870. In general, a nonhuman mammal is made transgenic for a human heavy chain locus and a human light chain locus, and the corresponding endogenous immunoglobulin loci are inactivated.

One group of soluble receptors that can be used as IL27 antagonists within the present invention comprises at least a ligand-binding portion of IL-27RA (Zcytor1 fragment) joined to a multimerizing protein as disclosed in Sledziewski et al., U.S. Pat. Nos. 5,155,027 and 5,567,584. Exemplary multimerizing proteins in this regard include immunoglobulin constant region domains. See also, Baumgartner et al., U.S. Pat. No. 5,792,850. Ig constant region domains may also be used to increase the circulatory half-life of fusion proteins comprising them or to add antibody-dependent effector functions. Fusion to an Fc fragment may also improve the production characteristics of a protein of interest. For example, an Zcytor1 fragment polypeptide comprising at least the cytokine-binding domain and up to the entire extracellular domain (approximately residues 33-514 of SEQ ID NO:5) can be joined to an IgG Fc fragment, including wild-type Fc fragments and engineered variants (including variants shown in FIGS. 1A-1C). In another embodiment, the C_{H}2 domain of the Fc fragment can be replaced with a linker peptide of approximately 15 amino acid residues. Such fusions are typically secreted as multimeric molecules wherein the Fc portions are disulfide bonded to each other and the two non-Ig polypeptides (e.g., receptor fragements) are arrayed in close proximity to each other. Immunoglobulin-IL-27RA polypeptide fusions can be expressed in genetically engineered cells to produce a variety of multimeric IL-27RA analogs. Within immunoglobulin-IL-27RA fusion proteins, certain amino acid subsititutions may be introduced into the Ig portion to alter effector functions associated with the native Ig. For example, amino acid substitutions can be made at EU index positions 234, 235, and 237 to reduce binding to Fc.gamma.RI, and at EU index positions 330 and 331 to reduce complement fixation. See, Duncan et al., Nature 332:563-564, 1988; Winter et al., U.S. Pat. No. 5,624,821; Tao et al., J. Exp. Med. 178:661, 1993; and Canfield and Morrison, J. Exp. Med. 173:1483, 1991. The carboxyl-terminal lysine residue can be removed from the C_{H}3 domain to increase homogeneity of the product. Within fusions to an Ig heavy chain polypeptide, the Cys residue within the hinge region that is ordinarily disulfide-bonded to the light chain can be replaced with another amino acid residue, such as a serine residue, if the Ig fusion is not co-expressed with a light chain polypeptide. However, an Ig-IL-27RA fusion polypeptide can be co-expressed with a wild-type or fused light chain polypeptide as disclosed in Sledziewski et al., U.S. Pat. No. 6,018,026.

As disclosed in more detail below, however, the inventors have found that a Zcytor1 fragment polypeptide joined to a wild-type murine Ig gamma2a Fc fragment was rapidly cleared from the circulation of experimental animals. In contrast, a fusion protein comprising an Fc fragment that had been engineered to remove effector functions (Fc5; FIGS. 1A-1C) showed a markedly improved circulatory half-life.

Proteins for use within the present invention can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., ibid.

In general, a DNA sequence encoding a protein of interest is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a recombinant protein into the secretory pathway of a host cell, a secretory signal sequence (also known as a leader sequence, prepro sequence or pre sequence) is provided in the expression vector. The secretory signal sequence may be that of IL-27RA itself, or may be derived from another secreted protein (e.g., t-PA; see, U.S. Pat. No. 5,641,655) or synthesized de novo. The secretory signal sequence is operably linked to the protein-encoding DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Expression of receptor-Fc fusion proteins via a host cell secretory pathway is expected to result in the production of multimeric (e.g., dimeric) proteins. If the fusion protein is to be produced as a dimer without associated immunoglobulin light chains, host cells that do not produce endogenous immunoglobulins are preferred as hosts, and the Fc portion of the fusion will preferably be modified to eliminate any unpaired cysteine residues. Multimers may also be assembled in vitro upon incubation of component polypeptides under suitable conditions. In general, in vitro assembly will include incubating the protein mixture under denaturing and reducing conditions followed by refolding and reoxidation of the polypeptides to form dimers. Recovery and assembly of proteins expressed in bacterial cells is disclosed below.

Cultured mammalian cells are suitable hosts for production of IL-27 antagonists. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., ibid.), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44; CHO DXB11 (Hyclone, Logan, Utah); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Va. Strong transcription promoters can be used, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny are referred to as “stable transfectants.” Exemplary selectable markers include a gene encoding resistance to the antibiotic neomycin, which allows selection to be carried out in the presence of a neomycin-type drug, such as G-418 or the like; the gpt gene for xanthine-guanine phosphoribosyl transferase, which permits host cell growth in the presence of mycophenolic acid/xanthine; and markers that provide resistance to zeocin, bleomycin, blastocidin, and hygromycin (see, e.g., Gatignol et al., Mol Gen. Genet. 207:342, 1987; Drocourt et al., Nucl. Acids Res. 18:4009, 1990). Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

Insect cells can be infected with recombinant baculovirus, commonly derived from Autographa californica nuclear polyhedrosis virus (AcNPV). See, King and Possee, The Baculovirus Expression System: A Laboratory Guide, Chapman & Hall, London; O'Reilly et al., Baculovirus Expression Vectors: A Laboratory Manual, Oxford University Press., New York, 1994; and Richardson, Ed., Baculovirus Expression Protocols. Methods in Molecular Biology, Humana Press, Totowa, N.J., 1995. Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, Md.). The transfer vector (e.g., PFASTBAC1; Life Technologies) contains a Tn7 transposon to move the DNA encoding the protein of interest into a baculovirus genome maintained in E. coli as a large plasmid called a “bacmid.” See, Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a protein-encoding DNA sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses the protein or interest is subsequently produced. Recombinant viral stocks are made by methods commonly used in the art.

For protein production, the recombinant virus is used to infect host cells, typically a cell line derived from the fall armyworm, Spodoptera frugiperda (e.g., Sf9 or Sf21 cells) or Trichoplusia ni (e.g., HIGH FIVE cells; Invitrogen, Carlsbad, Calif.). See, in general, Glick and Pasternak, Molecular Biotechnology Principles & Applications of Recombinant DNA, ASM Press, Washington, D.C., 1994. See also, U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0. 1 to 10, more typically near 3. Procedures used are generally described in available laboratory manuals (e.g., King and Possee, ibid.; O'Reilly et al., ibid.; Richardson, ibid.).

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936; and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii, and Candida maltosa are known in the art. See, for example, Gleeson et al., J. Gen. Microbiol. 132:3459-3465, 1986; Cregg, U.S. Pat. No. 4,882,279; and Raymond et al., Yeast 14:11-23, 1998. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a recombinant protein in bacteria such as E. coli, the protein may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured protein can then be refolded and dimerized by diluting the denaturant, such as by dialysis against a solution of urea and a combination of reduced and oxidized glutathione, followed by dialysis against a buffered saline solution. In the alternative, the protein may be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted proteins can be recovered from the periplasmic space in a soluble and functional form by disrupting the cells (by, for example, sonication or osmotic shock) to release the contents of the periplasmic space and recovering the protein, thereby obviating the need for denaturation and refolding. Antibodies, including single-chain antibodies, can be produced in bacterial host cells according to known methods. See, for example, Bird et al., Science 242:423-426, 1988; Huston et al. Proc. Natl. Acad. Sci. U.S.A. 85:5879-5883, 1988; and Pantoliano et al., Biochem. 30:10117-10125, 1991.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

IL-27 antagonist proteins are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, N.Y., 1994. Proteins comprising an immunoglobulin heavy chain polypeptide can be purified by affinity chromatography on immobilized protein A. Additional purification steps, such as gel filtration, can be used to obtain the desired level of purity or to provide for desalting, buffer exchange, and the like.

Antibodies can be purified from cell culture media by known methods, such as affinity chromatography using conventional columns and other equipment. In a typical procedure, conditioned medium is harvested and may be stored at 4° for up to five days. To avoid contamination, a bacteriostatic agent (e.g., sodium azide) is generally added. The pH of the medium is lowered (typically to pH≈5.5), such as by the addition of glacial acetic acid dropwise. The lower pH provides for optimal capture of IgG via a protein G resin. The protein G column size is determined based on the volume of the conditioned medium. The packed column is neutralized with a suitable buffer, such as 35 mM NaPO₄, 120 mM NaCl pH 7.2. The medium is then passed over the neutralized protein g resin at a flow rate determined by both the volume of the medium and of the column size. The flowthrough is retained for possible additional passes over the column. The resin with the captured antibody is then washed into the neutralizing buffer. The column is eluted into fractions using an acidic elution buffer, such as 0.1M glycine, pH 2.7 or equivalent. Each fraction is neutralized, such as with 2M tris, pH 8.0 at a 1:20 ratio tris:glycine. Protein containing fractions (e.g., based on A₂₈₀) are pooled. The pooled fractions are buffer exchanged into a suitable buffer, such as 35 mM NaPO₄, 120 mM NaCl pH 7.2 using a desalting column. Concentration is determined by A₂₈₀ using an extinction coefficient of 1.44. Endotoxin levels may be determined by LAL assay. Purified protein may be stored frozen, typically at −80° C.

For pharmaceutical use, IL-27 antagonists are formulated for topical or parenteral, particularly intravenous, intramuscular, or subcutaneous, delivery according to conventional methods. In general, pharmaceutical formulations will include an IL-27 antagonist in combination with a pharmaceutically acceptable vehicle, such as saline, buffered saline, 5% dextrose in water, or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. A “therapeutically effective amount” of a composition is that amount that produces a statistically significant effect, such as a statistically significant reduction in disease progression or a statistically significant improvement in organ function. Therapeutic endpoints for treatment of aplastic anemia include one or more of increased disease-free and overall survival, haematological response (increased numbers of blood cells, including platelets, neutrophils, and reticulocytes), reduction in symptoms (e.g., weakness, shortness of breath, palor, frequency or severity of infections, bleeding, and bruising), and prevention or reduction of relapse and late clonal complications (other haematological disorders such as paroxysmal nocturnal haemoglobinuria, myelodysplasia, or acute leukaemia). The exact dose will be determined by the clinician according to accepted standards, taking into account the nature and severity of the condition to be treated, patient traits, etc. Determination of dose is within the level of ordinary skill in the art. The therapeutic formulations will generally be administered over the period required to achieve a beneficial effect, commonly from several weeks up to several months and, in treatment of chronic conditions, for a year or more with periodic evaluations (e.g., at 3-month intervals) for clinical response. In patients known to be at risk for aplastic anemia (e.g., those receiving bone marrow transplants), the antagonists may be used prophylactically (e.g., beginning immediately post-transplant). Dosing is daily or intermittently (e.g., one, two, three, or more times per week) over the period of treatment. Intravenous administration will be by bolus injection or infusion over a typical period of one to several hours. Sustained release formulations can also be employed. An IL-27 antagonist may also be delivered by aerosolization according to methods known in the art. See, for example, Wang et al., U.S. Pat. No. 5,011,678; Gonda et al., U.S. Pat. No. 5,743,250; and Lloyd et al., U.S. Pat. No. 5,960,792.

A soluble receptor will commonly be administered at doses of 0.01 to 10 mg/kg of patient body weight, generally from 0.1 to 10 mg/kg, more often 1.0 to 10 mg/kg in multiple administrations (typically by injection or infusion) over a period of up to four weeks or more.

Efficacy of IL-27 antagonists is assayed in a mouse model of aplastic anemia (Bloom et al., Exp. Hematol. 32:1163-1172, 2004). Briefly, bone marrow failure and pancytopenia are induced in hybrid mice (B6D2F1 or CByB6F1) by infusion with lymph node cells from the C57B1/6 parent strain. IL-27 antagonists are administered, and the mice are monitored for pancytopenia by cell counting and for marrow failure by histological staining.

Antibodies are preferably administered parenterally, such as by bolus injection or infusion (intravenous, intramuscular, intraperitoneal, or subcutaneous) over the course of treatment. Antibodies are generally administered in an amount sufficient to provide a minimum circulating level of antibody throughout the treatment period of between approximately 20 .micro.g and 1 mg/kg body weight. In this regard, it is preferred to use antibodies having a circulating half-life of at least 12 hours, preferably at least 4 days, more preferably up to 14-21 days. Chimeric and humanized antibodies are expected to have circulatory half-lives of up to four and up to 14-21 days, respectively. In many cases it will be preferable to administer daily doses during a hospital stay, followed by less frequent (e.g., weekly) bolus injections during a period of outpatient treatment. An initial loading dose may be followed by lower maintenance doses. Antibodies can also be delivered by slow-release delivery systems, pumps, and other known delivery systems for continuous infusion. Dosing regimens may be varied to provide the desired circulating levels of a particular antibody based on its pharmacokinetics. Thus, doses will be calculated so that the desired circulating level of therapeutic agent is maintained. In general, doses of antibody will be in the range of 0.1 to 100 mg/kg, more commonly 0.5 to 20 mg/kg, and often 1.0 to 10 mg/kg depending on antibody pharmacokinetics and patient traits.

Within the present invention, an IL-27 antagonist can be administered in combination with one or more additional therapeutic agents, such as immunosuppressants (including steroids), chemotherapeutics, cytokine (e.g., IL-23, IL-6, IL-1, TNF-.alpha., or IL-12) antagonists (including antibodies and soluble receptors), hematopoietic agents (e.g., EPO, G-CSF, GM-CSF), antibiotics and other anti-infective drugs, and blood transfusions. Suitable IL-12 antagonists in this regard include anti-IL-12 antibodies (preferably targeting both the p40 and p35 subunits), anti-IL-12 receptor antibodies (preferably targeting both the IL-12R1 and IL-12R2 receptor subunits), and soluble IL-12 receptors. Soluble IL-12 receptors include soluble forms of IL-12R1, soluble forms of IL-12R2, and molecules comprising ligand-binding regions of both subunits, such as heterdimeric Ig fusion proteins and single-chain molecules comprising the two ligand-binding regions joined by a linker. IL-12 receptor subunits are disclosed by Chua et al., J Immunol. 153(1):128-136, 1994 and Presky et al., Proc. Natl. Acad. Sci. USA 93:14002-14007, 1996. Methods for producing bispecific antibodies are known in the art and are disclosed by, for example, Atwell et al. (ibid.) and Carter, J. Immunol. Methods 248:7-15, 2001.

Those skilled in the art will recognize that the same principles will guide the use of other IL-27 antagonists. The dosing regimen for a given antagonist will be determined by a number of factors including potency, pharmacokinetics, and the physicochemical nature of the antagonist.

The invention is further illustrated by the following non-limiting examples.

### EXAMPLES

### Example 1

Five 3 month old female CD rats (Charles River Laboratories, Wilmington, Mass.) were immunized with mouse IL-27RA (mIL-27RA). The rats were initially immunized by intraperitoneal injection with ˜50 .micro.g of purified, recombinant mouse IL-27RA-HIS (produced in CHO cells with a C-terminal HIS tag) in combination with a commercially available adjuvant (RIBI Adjuvant; Sigma-Aldrich, St. Louis, Mo.) according to the manufacturer's instructions. Following the initial immunization each of the rats received an additional 50 .micro.g of mIL-27RA in the same adjuvant via the intraperitoneal route every two weeks over a six-week period. Seven days after the third and fourth immunizations the rats were bled via the retroorbital plexus, and the serum was separated from the blood for analysis of its ability to bind to mIL-27RA in solution.

The ability of anti-mouse IL-27RA antibodies in the antisera to bind to mIL-27RA-HIS was assessed using a “capture” style ELISA assay. In this assay, wells of 96-well polystyrene ELISA plates were first coated with 100 .micro.L/well of goat anti-rat IgG, Fc-specific antibody (Jackson Immunoresearch) at a concentration of 1 μg/mL in Coating Buffer (0.1M Na₂CO₃, pH 9.6). Plates were incubated overnight at 4° C., after which unbound antibody was aspirated and the plates washed twice with 300 .micro.L/well of Wash Buffer (PBS-Tween, defined as 0.137M NaCl, 0.0027M KCl, 0.0072M Na₂HPO₄, 0.0015M KH₂PO₄, 0.05% v/v polysorbate 20, pH 7.2). Wells were blocked with 200 uL/well of Blocking Buffer (PBS-Tween plus 1% w/v bovine serum albumin (BSA)) for 60 minutes at room temperature, then buffer was aspirated from the wells and the plates were washed twice with 300 .micro.L/well of PBS-Tween. Serial 10-fold dilutions (in 1% BSA in PBS-Tween) of the sera were prepared beginning with an initial dilution of 1:1000 and ranging to 1:1,000,000. Duplicate samples of each dilution were then transferred to the assay plate, 100 uL/well, in order to bind rat IgG in the sera to the assay plate through the Fc portion of the molecule. Normal rat sera served as a negative control. Following a 1-hour incubation at room temperature, the buffer was aspirated from the wells, and the plates were washed twice as described above. Biotinylated mIL-27RA-HIS (3:1 molar ratio of biotin: protein) at a concentration of 100 ng/mL was then added to the wells, 100 .micro.L/well. Following a 1-hour incubation at room temperature, unbound biotinylated mIL-27RA-HIS was aspirated from the wells, and the plates were washed twice. Horseradish peroxidase-labeled streptavidin (“HRP-SA”) (Pierce, Rockford, Ill.) at a concentration of 500 ng/mL was then added to each well, 100 .micro.L/well, and the plates were incubated at room temperature for 1 hour. After removal of unbound HRP-SA, the plates were washed 5 times with 300 .micro.L/well of PBS-Tween. Tetramethyl benzidine (TMB) (BioFX Laboratories, Owings Mills, Md.) was then added to each well, 100 .micro.L/well, and the plates were incubated for 5 minutes at room temperature. Color development was stopped by the addition of 100 .micro.L/well of stop reagent (450 nm TMB Stop Reagent; BioFX Laboratories, Owings Mills, Md.), and the absorbance values of the wells were read on an absorbance microplate reader (SPECTRAMAX 340; Molecular Devices Corporation, Sunnyvale, Calif.) at 450 nm.

The ability of anti-mouse IL-27RA antibodies in the antisera to reduce the binding activity of IL-27RA to its cognate receptor was assessed using a plate-based neutralization ELISA. In this assay, wells of 96-well polystyrene ELISA plates were first coated with 100 .micro.L/well of a mouse IL-27RA-Fc fusion protein at a concentration of 1000 ng/mL in Coating Buffer. Plates were incubated overnight at 4° C., after which unbound receptor was removed by aspiration, and the plates were washed twice with 300 .micro.L/well of Wash Buffer. Wells were blocked with 200 .micro.L/well of Blocking Buffer for 1 hour, after which the plates were washed twice with Wash Buffer. Serial 10-fold dilutions (in 1% BSA in PBS-Tween) of the sera were prepared beginning with an initial dilution of 1:1000 and ranging to 1:1,000,000. Duplicate samples of each dilution were then transferred to the assay plate, 100 .micro.L/well, in order to bind rat IgG in the sera to the assay plate through the Fc portion of the molecule. Following a 1-hour incubation at room temperature, the wells were aspirated and the plates washed twice as described above. Biotinylated ligand (6:1 molar ratio of biotin:protein) at a concentration of 100 ng/ml was then added to the wells of the dilution plates, 100 .micro.L/well. Normal rat sera served as a negative control. Following a 1-hour incubation at room temperature, the wells were aspirated and the plates washed twice as described above. Horseradish peroxidase-labeled streptavidin (Pierce, Rockford, Ill.) at a concentration of 500 ng/mL was then added to each well, 100 .micro.L/well, and the plates were incubated at room temperature for 1 hour. After removal of unbound HRP-SA, the plates were washed twice with 300 .micro.L/well of PBS-Tween. TMB was then added to each well, 100 .micro.L/well, and the plates were incubated for 3 minutes at room temperature. Color development was stopped by the addition of 100 .micro.L/well of 450 nm stop reagent, and the absorbance values of the wells was read on an absorbance microplate reader at 450 nm.

Both the capture ELISA and the plate-based neutralization ELISA indicated that all five rats developed a significant antibody response to mIL-27RA. In general, the response as measured by the capture ELISA closely paralleled that seen with the plate-based neutralization ELISA, suggesting that IgG class antibody was primarily responsible for the inhibition of mIL-27RA.

### Example 2

Five and a half weeks after the last intraperitoneal immunization (Example 1), all rats were boosted with approximately 50 .micro.g of mIL-27RA-HIS with a commercially available adjuvant (RIBI Adjuvant; Sigma-Aldrich, St. Louis, Mo.). Two weeks after this boost, the rat with the most significant mIL-27RA titer was immunized a final time with approximately 50 .micro.g of mIL-27RA-HIS in PBS via intravascular injection. Five days later, the spleen and lymph nodes of this rat were harvested, prepared into a single cell suspension, and fused to the Ag8 mouse myeloma cell line at a 2:1 lymphoid cell:myeloma cell ratio with PEG 1500 using standard methods (Harlow and Lane, ibid.). The fusion mixture was distributed into 20 96-well flat-bottomed plates in combination with BALB/c thymocytes as a feeder layer (Oi and Herzenberg in “Selected Methods in Cellular Immunology” Mishell and Shiigi, eds., pp. 351-372, Freeman, San Francisco, 1980). Wells of the fusion plates were fed three times with a 70% replacement of media. Wells were assayed ten days after plating of the fusion. This fusion was designated “Fusion 290.”

For a second fusion, approximately 3 months after the last intraperitoneal immunization (Example 1), all remaining rats were boosted with approximately 50 .micro.g of mIL-27RA-HIS with a commercially available adjuvant (RIBI Adjuvant; Sigma-Aldrich, St. Louis, Mo.). Four weeks after this boost, the rat with the most significant mIL27RA neutralizing titer was immunized a final time with approximately 50 .micro.g of mIL-27RA-HIS in PBS via intravascular injection. Five days later, the spleen and lymph nodes of this rat were harvested, prepared into a single cell suspension, and fused to the Ag8 mouse myeloma cell line at a 2:1 lymphoid cell:myeloma cell ratio with PEG 1500 using standard methods. The fusion mixture was distributed into 15 96-well flat-bottomed plates. Wells of the fusion plates were fed three times with a 70% replacement of media. Wells were assayed ten days after plating of the fusion. This fusion was designated “Fusion 295.”

The capture ELISA for mIL-27RA as disclosed in Example 1 was used as the primary screen for Fusion 290 except that hybridoma supernatants were tested undiluted from the culture plates. Approximately 290 positive wells were identified. Hybridoma cells from positive wells were expanded into culture in 24-well plates. When the density of the 24-well cultures was approximately 4-6×10⁵ cells/mL, the supernatants (approximately 1.5 mL each) were individually collected and stored, and the cells from each well were cryopreserved. Supernatants from each of these wells as well as a few negative wells were then assessed for their ability to inhibit mIL27RA in the plate-based neutralization assay disclosed in Example 1. Nine of the supernatants appeared to neutralize mIL27RA.

The neutralization ELISA for mIL-27RA (Example 1) was used as the primary screen for Fusion 295 except that hybridoma supernatants were tested undiluted from the culture plates. Twenty positive wells were identified for further evaluation. Hybridoma cells from the positive wells were expanded into culture in 24-well plates. When the density of the 24-well cultures was approximately 4-6×10⁵ cells/mL, the supernatants (approximately 1.5 mL each) were individually collected and stored, and the cells from each well were cryopreserved.

Each of the 24-well supernatants was reanalyzed in both the capture ELISA and plate-based neutralization ELISA. Results indicated that following expansion, all of the master well supernatants had retained their ability to recognize mouse IL-27RA in solution. The majority of the master well supernatants retained their ability to neutralize mouse IL-27RA.

Cells in six of the IL-27RA neutralizing master wells (290.118.6, 290.267.1, 295.6.4, 295.13.4, 295.16.2, and 295.20.4) were cloned in order to isolate a cloned hybridoma producing a neutralizing monoclonal antibody of interest. Cells were cloned in 96-well microtiter cell culture plates using a standard low-density dilution (less than 1 cell per well) approach, and monoclonality was assessed by microscopic examination of wells for a single focus of growth prior to assay. Six days post-plating, all wells on the plates were screened by the neutralization ELISA. Supernatant from approximately 6 wells that was both positive for specific mAb and originated from wells with only a single colony of hybridoma growth was collected from each cloning set and rescreened at various dilutions in the neutralization ELISA to identify a “best” neutralizing mAb-producing clone. A “best” clone in each of these sets was recloned, and the subclones were screened as described above to yield the final hybridoma lines 290.118.6.6, 290.267.1.4, 295.6.4.6, 295.13.4.1, 295.16.2.1, and 295.20.4.3. The rat IgG isotype of the mAb produced by each of these hybridomas was determined using an ELISA that employed biotinylated anti-rat IgG isotype specific mAbs. All six mAbs were found to belong to the IgG1 (290.267.1.4, 295.13.4.1, 295.16.2.1, and 295.20.4.3) or IgG2a (290.118.6.6 and 295.6.4.6) subclasses.

Characterization of anti-IL-27RA antibodies is shown in Table 1. Epitope “bin” numbers were assigned by competition binding experiments; antibodies found to compete for binding were assigned to the same bin. Binding affinity (Kd) was determined by surface plasmon resonance on an automated instrument (BIACORE 3000; Biacore International AB, Uppsala, Sweden) using standard protocols. EC50 (amount of antibody needed to obtain 50% positive signal) was determined by ELISA. IC50 values were determined using the spleen/STAT3 bioassay essentially as disclosed in Example 5; data were obtained from triplicate experiments using first round-clones from which the indicated second-round clones were derived. Cell depletion was determined experimentally in mice (3/group) injected on days 0, 1 and 2 intraperitoneally with either PBS, anti-CD4 mAb, rat isotype control mAb (IgG1 or IgG2a), or one of the indicated anti-IL-27RA mAbs (0.5 mg/mouse of mAb in 0.5 ml PBS). Mice were sacrificed on day 6. Single-cell suspensions of spleen, lymph-node, thymus, and bone-marrow cells were prepared and stained for 8-color flow-cytometry analysis. To detect cell-bound mAbs, the cells were co-stained with an anti-CD3 mAb (2C11-PE/Cy7; BD-PHARMINGEN; BD Biosciences, San Diego, Calif.) and APC-labeled donkey-anti-rat IgG polyclonal antibody (obtained from eBioscience, San Diego, Calif.). For comparative purposes, cells from PBS-treated mice were stained with the neutralizing mAbs before staining with anti-CD3 and anti-rat IgG. Spleen, thymus and lymph-node cells were stained with mAbs specific for CD44, CD62L, CD69, CD3, CD8, CD49, CD25 and CD4 to identify T cell subpopulations, NKT cells and NK cells. Spleen and lymph-node cells were stained with mAbs specific for CD23, CD21, CD11 b, IgM, IgD, CD11c, Gr-1 and B220 to identify B cell subpopulations, granulocytes, macrophages and dendritic cells. Bone marrow cells were stained for IgD, CD43, CD11b, IgM, B220, CD11c and Gr-1 to identify B cell subpopulations, macrophages, dendritic cells and granulocytes. The flow-cytometry data (100,000 events/sample) was analyzed using commercially available software (FACS DIVA, Becton-Dickinson). All mice treated with IL-27RA neutralizing mAbs had a saturating level of neutralizing mAb bound to their T cells. None of the various immune populations analyzed was depleted after treatment with PBS, rat isotype control mAb or the IL-27RA neutralizing mAbs. The anti-CD4 mAb depleted >95% of the CD4 T cells in all mice treated with this mAb, thus serving as positive control. FACS analysis was carried out on C57B1/6 mouse spleen cells stained in duplicate with graded concentrations (range=0 to 20 .micro.g/ml) of each mAb, then washed and stained with PE/Cy7-labeled anti-CD3 mAb (BD PHARMINGEN) and APC-labeled anti-rat IgG polyclonal antibody (eBioscience) for 1 hour on ice, and analyzed by flow cytometry. Mean fluorescence intensity (MFI) of IL-27RA-APC staining on CD3-postive lymphocytes was compared.

TABLE 1

| | | | | | | |
|---|---|---|---|---|---|---|
| mAb: | 290.118.6.6 | 295.6.4.6 | 295.16.2.1 | 295.20.4.3 | 290.267.1.4 | 295.13.4.1 |
| Epitope bin: | 1 | 1 | 1 | 2 | 2 | 3a |
| Isotype: | IgG2a/kappa | IgG2a/kappa | IgG1/kappa | IgG1/kappa | IgG1/kappa | IgG1/kappa |
| Binding affinity: | 0.45 nM | 1.3 nM | 3.7 nM | 1.40 nM | | |
| EC50: | 0.38 nM | 0.33 nM | 0.64 nM | 0.53 nM | 0.61 nM | 0.40 nM |
| IC50 (nM): | 0.72, 0.52, | 0.46, 0.34, | 0.21, 0.37, | 0.17, 0.54, | unclear | unclear |
| | 0.64 | 0.31 | 0.24 | 0.55 | | |
| Cell-depleting | No | No (??) | No | No | | |
| in vivo: | | | | | | |
| FACS staining: | Best | Best | Moderate | Moderate | Moderate | Moderate |
| Cross-reacts | No | No | No | No | No | No |
| w/human: | | | | | | |

### Example 3

A DNA construct encoding a fusion protein (designated “IL27RAm(mFc1)”) comprising the extracellular domain of mouse IL27RA and a wild type BALB/c mouse .gamma.2a constant region Fc tag was constructed via a 3-step PCR and homologous recombination using a DNA fragment encoding the extracellular domain of mouse IL27RA and the expression vector pZMP40. Plasmid pZMP40 is a mammalian expression vector containing an expression cassette comprising the chimeric CMV enhancer/MPSV promoter, a BglII site for linearization prior to yeast recombination, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae. pZMP40 is a derivative of plasmid pZMP21, which is described in U.S. patent application publication No. 2003/0232414 A1 and has been deposited at the American Type Culture Collection, 10801 University Boulevard, Manassas, Va. 20110-2209, designated No. PTA-5266.

A PCR fragment encoding IL27RAm(mFc1) was constructed to contain a 5′ overlap with the pZMP40 vector sequence in the 5′ non-translated region, the IL27RA extracellular domain coding region, the C-terminal mFc1 tag coding sequence, and a 3′ overlap with the pZMP40 vector in the poliovirus internal ribosome entry site region. The first PCR amplification reaction used the 5′ oligonucleotide primer zc46250 (SEQ ID NO:14), the 3′ oligonucleotide primer zc47631 (SEQ ID NO:15), and a previously generated plasmid containing mouse IL27RA cDNA as the template. A second PCR fragment was generated using the 5′ oligonucleotide primer zc24901 (SEQ ID NO:16), the 3′ oligonucleotide primer zc46896 (SEQ ID NO:17) and a previously generated plasmid containing mouse Fc cDNA as the template. The PCR amplification reaction conditions were as follows: One cycle of 95° C. for 5 minutes; then 35 cycles of 95° C. for 30 seconds, 55° C. for 30 seconds, and 68° C. for 2 minutes; then one cycle of 68° C. for 10 minutes; followed by a 4° C. hold. The PCR reaction mixtures were run on a 1.2% agarose gel, and the DNA fragments corresponding to the expected size were extracted from the gel using a commercially available gel extraction kit (QIAQUICK Gel Extraction Kit; QIAGEN Inc., Valencia, Calif.).

The two fragments were then joined and amplified using the 5′ oligonucleotide primer zc46250 (SEQ ID NO: 14) and the 3′ oligonucleotide primer zc46759 (SEQ ID NO: 18) under the following PCR conditions: one cycle of 95° C. for 3 minutes; then 35 cycles of 95° C. for 30 seconds and 72° C. for 2 minutes; then one cycle of 72° C. for 7 minutes; followed by a 4° C. hold. The final PCR product was cloned using a commercially available kit (TOPO TA CLONING Kit; Invitrogen, Carlsbad, Calif.) according to the manufacturer's directions. Two μL of the cloning reaction mixture was used to transform chemically competent E. coli cells (ONE SHOT DH10B-T1; Invitrogen), which were plated onto LB AMP plates (LB broth (Lennox), 1.8% BACTO Agar (DIFCO), 100 mg/L Ampicillin) overnight. Colonies were sequenced and found to have deletions within the IL27RA coding region. This discrepancy was resolved by performing a double digest with KpnI and SpeI on two clones and ligating the two correct fragments using a commercially available DNA ligation kit (FAST-LINK; EPICENTRE Biotechnologies, Madison, Wis.) according to the manufacturer's protocol. A resulting colony that contained the corrected insert sequence was grown up in LB AMP broth, and the plasmid was purified with a commercially available kit (QIAPREP Spin Miniprep kit; QIAGEN Inc.). The plasmid clone was then digested with EcoRI, and the IL27RAm(mFc1) insert was excised and purified using a commercially available gel extraction kit (QIAQUICK Gel Extraction Kit).

The plasmid pZMP40 was digested with BglII prior to recombination in yeast with the purified IL27RAm(mFc1) fragment. One hundred μL of competent yeast (S. cerevisiae) cells were combined with 10 μL (1 .micro.g) of the IL27RAm(mFc1) insert DNA and 100 ng of BglII-digested pZMP40 vector, and the mixture was transferred to a 0.2-cm electroporation cuvette. The yeast/DNA mixture was electropulsed using power supply (BIORAD Laboratories, Hercules, Calif.) settings of 0.75 kV (5 kV/cm), ∞ ohms, and 25 μF. Six hundred μL of 1.2 M sorbitol was added to the cuvette, and the yeast was plated in 300-μL aliquots onto two URA-D plates (U.S. Pat. No. 5,736,383) and incubated at 30° C. After about 72 hours, the Ura⁺ yeast transformants from a single plate were resuspended in 1 ml H₂O and spun briefly to pellet the yeast cells. The cell pellet was resuspended in 500 μL of lysis buffer (2% t-octylphenoxypolyethoxyethanol (TRITON X-100), 1% SDS, 100 mM NaCl, 10 mM Tris, pH 8.0, 1 mM EDTA). The 500 μL of the lysis mixture was added to a microcentrifuge tube containing 300 μL acid-washed glass beads and 200 μL phenol-chloroform, vortexed for 2 minutes, and spun for 5 minutes in a microcentrifuge at maximum speed. Three hundred μL of the aqueous phase was transferred to a fresh tube, and the DNA was precipitated with 600 μL ethanol, followed by centrifugation for 10 minutes at maximum speed. The tube was decanted, and the DNA pellet was resuspended in 10 μL deionized H₂O.

Transformation of electrocompetent E. coli host cells (DH10B) was performed using one μL of the yeast DNA preparation and 25 μl of E. coli cells. The cells were electropulsed at 2.5 kV, 25 μF, and 200 ohms. Following electroporation, 1 ml SOC (2% BACTO Tryptone (DIFCO, Detroit, Mich.), 0.5% yeast extract (DIFCO), 10 mM NaCl, 2.5 mM KCl, 10 mM MgCl₂, 10 mM MgSO₄, 20 mM glucose) was added, and the cells were plated in 100-μL and 500-μL aliquots on two LB AMP plates. The inserts of three DNA clones for the construct were subjected to sequence analysis, and one clone containing the correct sequence was selected. Large-scale plasmid DNA was isolated using a commercially available kit (QIAGEN ENDOFREE Plasmid Mega Kit; QIAGEN Inc.) according to the manufacturer's instructions. The sequence of the insert DNA is shown in SEQ ID NO: 19.

For transfection into CHO cells, 600 μg of the IL27RAm(mFc1)/pZMP40 expression plasmid was digested with 600 units of BstB1 at 37° C. for three hours, purified via phenol-chloroform extraction, and aliquoted to three microcentrifuge tubes. 0.1 volume 3M NaOAC, pH 5.2, and 2.2 volumes ethanol were added to each tube, and the tubes were stored on ice until transfection. The DNA was then spun down in a microfuge for 10 minutes at 14,000 RPM, and the supernatant was decanted off each pellet. The pellets were washed with 70% ethanol, decanted, and allowed to air dry for 15 minutes, then resuspended in 200 μL each of CHO cell culture medium in a sterile environment and allowed to incubate at 37° C. until the DNA pellets dissolved. Three tubes of approximately 1×10⁷ CHO DXB11 cells from log-phase culture were pelleted and resuspended in 600 μL warm medium. The DNA/cell mixtures were combined and placed in three 0.4-cm gap cuvettes and electroporated at 950 μF, high capacitance, 300 V. The contents of each cuvette was removed and diluted to 20 mL with CHO cell culture medium and placed in a 125-mL shake flask. The flasks were placed in a 37° C., 5% CO₂ incubator on a shaker platform set at 120 RPM. After approximately 48 hours, the contents of the three flasks were pooled and subjected to nutrient selection and step amplification to 200 nM methotrexate (MTX), and then to 1 μM MTX. Tagged protein expression was confirmed by Western blot, and the CHO cell pool was scaled-up for harvests for protein purification.

### Example 4

An expression plasmid encoding a human IL27RA-Fc5 fusion protein was constructed via homologous recombination in yeast. DNA fragments encoding the extracellular domain and secretion leader peptide of human IL27RA (amino acids 1 to 512 of SEQ ID NO:5) and Fc5 were inserted into the mammalian expression vector pZMP42. Fc5 is an effector minus form of human gamma1 Fc (FIGS. 1A-1C). pZMP42 is a derivative of plasmid pZMP21, made by eliminating the hGH polyadenylation site and SV40 promoter/dhfr gene and adding an HCV IRES/dhfr to the primary transcript, making it tricistronic.

The indicated fragment of IL27RA cDNA (nucleotides 23-1558 of SEQ ID NO:4) was isolated using PCR. The upstream primer for PCR (zc53405; SEQ ID NO:21) included, from 5′ to 3′ end, 37 bp of flanking sequence from the vector and 21 bp corresponding to the amino terminus from the open reading frame of IL27RA. The downstream primer (zc51828; SEQ ID NO:22) consisted of, from 5′ to 3′, 39 bp of the bottom strand sequence of Fc5 fusion protein sequence and the last 24 bp of the IL27RA extracellular domain sequence, nucleotides 1538 to 1558 of SEQ ID NO:4.

The Fc5 moiety was made with an upstream primer (zc51827; SEQ ID NO:23) including, from 5′ to 3′, 39 bp of flanking sequence from the IL27RA extracellular domain sequence and 24 bp corresponding to the sequence for the amino terminus of the Fc5 partner. The downstream primer for the Fc5 portion of the fusion protein (zc42508; SEQ ID NO:24) consisted of, from 5′ to 3′, 42 bp of the flanking sequence from the vector, pZMP42, and the last 20 bp of the Fc5 sequence.

The PCR amplification reaction conditions were 1 cycle, 94° C., 5 minutes; 25 cycles, 94° C., 1 minute, followed by 65° C., 1 minute, followed by 72° C., 1 minute; 1 cycle, 72° C., 5 minutes. Ten μL of each 100-μL PCR reaction mixture was run on a 0.8% low melting temperature agarose gel (SEAPLAQUE GTG) with 1×TBE buffer (0.892M Tris Base, 0.0223M EDTA, 0.890M boric acid) for analysis. The plasmid pZMP42, which had been cut with Bg1II, was used for homologous recombination with the PCR fragments. The remaining 90 μL of each PCR reaction and 200 ng of cut pZMP42 was precipitated with the addition of 20 μL 3 M Na Acetate and 500 μL of absolute ethanol, rinsed, dried and resuspended in 10 .micro.L water.

One hundred .micro.L of competent yeast cells (S. cerevisiae) was combined with 10 μL of the DNA mixture from above and transferred to a 0.2-cm electroporation cuvette. The yeast/DNA mixtures were electropulsed at 0.75 kV (5 kV/cm), ∞ ohms, 25 μF. To each cuvette was added 600 .micro.L of 1.2 M sorbitol, and the yeast was plated in two 300-μL aliquots onto two URA-D plates (U.S. Pat. No. 5,736,383) and incubated at 30° C. After about 48 hours, approximately 50 .micro.L packed yeast cells taken from the Ura+ yeast transformants of a single plate was resuspended in 100 .micro.L of lysis buffer (Example 3), 100 .micro.L of resuspension buffer (Buffer P1; QIAGEN Inc., Valencia, Calif.) and 20 U of a β-1,3-glucan laminaripentaohydrolase and b-1,3-glucanase (ZYMOLYASE; Zymo Research, Orange, Calif.). This mixture was incubated for 30 minutes at 37° C., and the remainder of the miniprep protocol (QIAGEN Inc.) was performed. The plasmid DNA was eluted twice in 100 μL water and precipitated with 20 μL 3 M Na Acetate and 500 μL absolute ethanol. The pellet was rinsed once with 70% ethanol, air-dried, and resuspended in 10 μL water for transformation.

Fifty μL electrocompetent E. coli cells (DH10B, Invitrogen, Carlsbad, Calif.) was transformed with 2 μL yeast DNA. The cells were electropulsed at 1.7 kV, 25 μF and 400 ohms. Following electroporation, 1 ml SOC (2% BACTO Tryptone (DIFCO, Detroit, Mich.), 0.5% yeast extract (DIFCO), 10 mM NaCl, 2.5 mM KCl, 10 mM MgCl₂, 10 mM MgSO₄, 20 mM glucose) was plated in 250, 100 and 10 μl aliquots on three LB AMP plates.

Individual clones harboring the correct expression construct for IL27RA-Fc5 were identified by restriction digest to verify the presence of the insert and to confirm that the various DNA sequences had been joined correctly to one another. The inserts of positive clones were subjected to sequence analysis. Larger scale plasmid DNA was isolated using a commercially available kit (QIAGEN Maxi kit; QIAGEN Inc., Valencia, Calif.) according to the manufacturer's instructions. DNA and amino acid sequence for IL-27RA-Fc5 are shown in SEQ ID NOS:2 and 3.

Three sets of 200 μg of the IL27RA-Fc5 constructs were separately digested with 200 units of PvuI at 37° C. for three hours, precipitated with ethanol, and centrifuged in a 1.5-mL microfuge tube. The supernatant was decanted off the pellet, and the pellet was washed with 300 μL of 70% ethanol and allowed to incubate for 5 minutes at room temperature. The tube was spun in a microfuge for 10 minutes at 14,000 RPM, and the supernatant was decanted off the pellet. The pellet was then resuspended in 750 μl of CHO cell tissue culture medium in a sterile environment, allowed to incubate at 60° C. for 30 minutes, then allowed to cool to room temperature. Approximately 5×10⁶ CHO cells were pelleted in each of three tubes and resuspended using the DNA-medium solution. The DNA/cell mixtures were placed in a 0.4-cm gap cuvette and electroporated at 950 μF, high capacitance, 300 V. The contents of the cuvettes were then removed, pooled, and diluted to 25 mL with CHO cell tissue culture medium and placed in a 125-mL shake flask. The flask was placed in an incubator on a shaker at 37° C., 6% CO₂ with shaking at 120 RPM.

The CHO cells were subjected to nutrient selection followed by step amplification to 200 nM methotrexate (MTX), and then to 1 μM MTX. Tagged protein expression was confirmed by Western blot, and the CHO cell pool was scaled-up for harvests for protein purification.

To purify the fusion protein, 10 L of conditioned media were harvested, sterile filtered using 0.2 μm filters, and adjusted to pH 7.2. The protein was purified from the filtered media using a combination of affinity chromatography on protein A and size-exclusion chromatography. A 117-ml (50 mm×60 mm) protein A column (POROS A50 Applied Biosciences, Foster City, Calif.) was pre-eluted with 3 column volumes (CV) of 25 mM sodium citrate—sodium phosphate, 250 mM ammonium sulfate pH 3 buffer and equilibrated with 20 CV PBS. Direct loading to the column at 31 cm/hr overnight at 4° C. captured the IL27RA-Fc5 in the conditioned media. After loading was complete, the column was washed with 10 CV of equilibration buffer. The column was then washed with 10 CV of 25 mM sodium citrate—sodium phosphate, 250 mM ammonium sulfate pH 7.2 buffer, then the bound protein was eluted at 92 cm/hr with a 20 CV gradient from pH 7.2 to pH 3 formed using the citrate-phosphate-ammonium sulfate buffers. Fractions of 10 ml each were collected into tubes containing 500 μl of 2.0 M Tris, pH 8.0 in order to neutralize the eluted proteins. The fractions were pooled based on A₂₈₀ and non-reducing SDS-PAGE.

The IL27RA-Fc5-containing pool was concentrated to 10 ml by ultrafiltration using centrifugal membrane filters (AMICON Ultra-15 30K NWML centrifugal devices; Millipore Corporation, Billerica, Mass.) and injected onto a 318-ml (26 mm×600 mm) size-exclusion chromatography column (SUPERDEX 200 GE Healthcare, Piscataway, N.J.) pre-equilibrated in 35 mM sodium phosphate, 120 mM NaCl pH 7.3 at 28 cm/hr. The fractions containing purified IL27RA-Fc5 were pooled based on A₂₈₀ and SDS PAGE, filtered through a 0.2-μm filter, and frozen as aliquots at −80° C. The concentration of the final purified protein was determined by calorimetric assay (BCA assay; Pierce, Rockford, Ill.). The overall process recovery was approximately 80%.

Recombinant IL27RA-Fc5 was analyzed by SDS-PAGE (4-12% BisTris, Invitrogen, Carlsbad, Calif.) with 0.1% Coomassie R250 staining for protein and immunoblotting with Anti-IgG-HRP. The purified protein was electrophoresed and transferred to nitrocellulose (0.2 μm; Invitrogen, Carlsbad, Calif.) at ambient temperature at 600 mA for 45 minutes in a buffer containing 25 mM Tris base, 200 mM glycine, and 20% methanol. The filters were then blocked with 10% non-fat dry milk in 50 mM Tris, 150 mM NaCl, 5 mM EDTA, 0.05% Igepa1 (TBS) for 15 minutes at room temperature. The nitrocellulose was quickly rinsed, and the IgG-HRP antibody (1:10,000) was added. The blots were incubated overnight at 4° C., with gentle shaking. Following the incubation, the blots were washed three times for 10 minutes each in TBS, and then quickly rinsed in H₂O. The blots were developed using commercially available chemiluminescent substrate reagents (LUMILIGHT; Roche), and the signal was captured using commercially available software (Lumi-Imager's Lumi Analyst 3.0; Boehringer Mannheim GmbH, Germany). The purified IL27RA-Fc5 appeared as a band at about 200 kDA on both the non-reducing Coomassie-stained gel and on the immunoblot, suggesting a glycosylated dimeric form as expected. Size-exclusion chromatography/multi-angle light scattering (SEC MALS) confirmed a mass consistent with a dimer containing additional mass contribution from carbohydrate at approximately 27% by weight, for a total mass of 212 kD (+/−5%). The protein had the correct NH₂ terminus and the correct amino acid composition.

### Example 5

Whole mouse spleens were harvested from C57 B1/6 mice and washed two times with 1X PBS before being plated out at 2×10⁵ cells/well in assay media (RPMI 1640 plus 10% fetal bovine serum) in 96-well, round-bottom tissue culture plates. Leukocytes (white blood cells) were thawed from a frozen vial collected from a leukapherisis donation and washed two times with 1X PBS before being plated out at 10⁶ cells/well in assay media in 96-well, round-bottom tissue culture plates. A sub-maximal concentration (EC₉₀, effective concentration at 90 percent) of mouse IL-27 (muIL-27) and human IL-27 (huIL-27) were each combined with a dose range of the human IL-27RA and mouse IL-27RA soluble receptors (Fc fusions) and incubated together at 37° C. for 30 minutes in assay media prior to addition to cells. Following pre-incubation, treatments were added to the plates containing the cells and incubated together at 37° C. for 15 minutes.

Following incubation, cells were washed with ice-cold wash buffer (BIO-PLEX Cell Lysis Kit, BIO-RAD Laboratories, Hercules, Calif.) and put on ice to stop the reaction according to manufacturer's instructions. Cells were then spun down at 2000 rpm at 4° C. for 5 minutes prior to dumping the media. 50 μL/well lysis buffer was added to each well; lysates were pipetted up and down five times while on ice, then agitated on a microplate platform shaker for 20 minutes at 300 rpm and 4° C. Plates were centrifuged at 4500 rpm at 4° C. for 20 minutes. Supernatants were collected and transferred to a new microtiter plate for storage at −20° C.

Capture beads (BIO-PLEX Phospho-Stat3 Assay, BIO-RAD Laboratories) were combined with 50 μL of 1:1 diluted lysates and added to a 96-well filter plate according to manufacture's instructions (BIO-PLEX Phosphoprotein Detection Kit, BIO-RAD Laboratories). The aluminum foil-covered plate was incubated overnight at room temperature with shaking at 300 rpm. The plate was transferred to a microtiter vacuum apparatus and washed three times with wash buffer. After addition of 25 μL/well detection antibody, the foil-covered plate was incubated at room temperature for 30 minutes with shaking at 300 rpm. The plate was filtered and washed three times with wash buffer. Streptavidin-PE (50 μL/well) was added, and the foil-covered plate was incubated at room temperature for 15 minutes with shaking at 300 rpm. The plate was filtered and washed two times with bead resuspension buffer. After the final wash, beads were resuspended in 125 μL/well of bead suspension buffer, shaken for 30 seconds, and read on an array reader (BIO-PLEX, BIO-RAD Laboratories) according to the manufacture's instructions. Data were analyzed using analytical software (BIO-PLEX MANAGER 3.0, BIO-RAD Laboratories). Decreases in the level of the phosphorylated STAT3 transcription factor present in the lysates were indicative of neutralization of the IL-27 receptor-ligand interaction.

For mouse spleens, muIL-27 EC₉₀ concentration was determined to be 0.2 nM and huIL-27 to be 2 nM. For total human PBMCs, both mouse and human IL-27 EC₉₀ concentrations were 2 nM. Run in combination with a dose-response of the mouse IL-27RA or human IL-27RA soluble receptor, the IC₅₀ (inhibitory concentration at 50%) was determined for each soluble receptor to each ligand on both cell types. Data are shown in Tables 2 and 3.

TABLE 2
Mouse Spleens

| Ligand | Soluble Receptor | IC₅₀ (nM) |
|---|---|---|
| muIL-27 | IL-27RAm(mFc1) | 0.18 |
| muIL-27 | human IL-27RA-Fc5 | 0.14 |
| huIL-27 | IL-27RAm(mFc1) | 9.30 |
| huIL-27 | human IL-27RA-Fc5 | 0.32 |

TABLE 3
Total Human PBMCs

| Ligand | Soluble Receptor | IC₅₀ (nM) |
|---|---|---|
| muIL-27 | IL-27RAm(mFc1) | 4.83 |
| muIL-27 | human IL-27RA-Fc5 | 2.97 |
| huIL-27 | IL-27RAm(mFc1) | 1370 |
| huIL-27 | human IL-27RA-Fc5 | 0.95 |

### Example 6

Kinetic rate and affinity constant values for the mouse (IL27RAm(mFc1), Example 3) and human (IL27RA-Fc5, Example 4) soluble receptors were obtained by surface plasmon resonance (SPR) using an automated instrument (BIACORE 3000; Biacore International AB, Uppsala, Sweden). The mouse soluble receptor was tested against mouse ligand (lot A1418F), and the human soluble receptor was tested against both mouse (A1426F) and human (A1534F) ligands. For determination of the kinetic rate constants for the receptor-ligand interactions, the gp130 molecule was not included as part of the receptor complex. Experimental evidence indicated that gp130 did not play a role in the binding mechanism, but affected only signaling (i.e., subsequent generation of physiological response), hence the measurement of the interaction between IL27RA and IL27 ligand was expected to accurately assess the affinity of simple binding of the ligand to its receptor.

The IL27 ligands used in this study were single-chain molecules comprising EBI3 connected by its C-terminus to the N-terminus of IL-27 p28 via a polypeptide linker. Each of the ligands included an amino-terminal peptide tag.

For the mouse IL27RA study, the soluble receptor was captured onto the chip surface by an isotype-specific anti-mouse Fc antibody (obtained from Jackson ImmunoResearch Laboratories, Inc., West Grove, Pa.) covalently immobilized to the chip (BIACORE CM5 chip) using the standard amine coupling protocol specified by the instrument manufacturer. For the human IL27RA studies, the soluble receptor was directly and covalently immobilized to the chip via the amine coupling protocol. In all studies, ligand was injected over the active (receptor-bound) surface at varying concentrations to obtain a series of binding curves.

Experimental conditions were optimized for determination of kinetic rate constant values. The molecular densities of the soluble receptor proteins loaded onto the chip surface were targeted to obtain maximum IL27 binding levels (Rₘₐₓ) of ≦20 RU. The analyte (ligand) was injected over the receptor surface at a flow rate of 50 μL/minute at a concentration range of approximately 0.05 to 10 nM, allowing for an association phase of 3 minutes and a dissociation phase of 10 minutes. The mouse soluble receptor surface was regenerated with two 30-second injections at 50 μL/minute of glycine, pH 2.0. The human soluble receptor surface was similarly regenerated with a single 30-second injection.

All data were assessed using software provided with the instrument (BIACORE Evaluation software v. 3.2). The binding curves were globally fitted to a 1:1 binding model corrected for mass transport limitation resulting from the fast on-rate values (kₐ) obtained. Statistical analysis of the fits of the experimental binding curves versus theoretical curves gave standard error values for kₐ and k_{d} of less than 2%, and chi² values of less than 2% of Rₘₐₓ for all interactions tested, providing reasonable confidence in the kinetic rate constant values obtained.

The kinetic rate and affinity constants obtained for mouse soluble receptor binding with mouse ligand were kₐ=1.0×10⁷ (M⁻¹s⁻¹), k_{d}=1.2×10⁻³ (S^{31 1}) and K_{d}=1.2×10⁻¹⁰ M (K_{d}=k_{d}/kₐ). The kinetic rate and affinity constants obtained for human soluble receptor binding with human ligand were kₐ=1.0×10⁷ (M⁻¹s⁻¹), k_{d}=1.9×10⁻³ (s⁻¹) and K_{d}=1.9×10⁻¹⁰ M. The kinetic rate and affinity constants obtained for human soluble receptor binding with mouse ligand were kₐ=8.1×10⁶ (M⁻¹s⁻¹), k_{d}=1.8×10⁻³ (s⁻¹) and K_{d}=2.2×10⁻¹⁰ M.

### Example 7

Studies were performed to evaluate the pharmacokinetics of the mouse (IL-27RAm(mFc1)) and human (IL-27RA-Fc5) soluble receptors in female C57B1/6 mice. Mice were randomly assigned to treatment groups as shown in Table 4.

TABLE 4

| | Route of | Dose | Sample Time Points |
|---|---|---|---|
| Treatment | Admin. | (μg) | (hours postdose) |
| IL-27RAm(mFc1) | IV | 100 | 0.25, 1, 3, 6, 24, 48, & 120 |
| | IP | 100 | |
| | SC | 100 | |
| IL-27RA-Fc5 | IV | 100 | 0.25, 0.5, 1, 3, 6, 24, 48, & 120 |
| | IP | 100 | |
| | SC | 100 | |

Whole blood was collected at the time points listed in Table 4. Serum was generated from each sample and analyzed by a qualified enzyme-linked immunosorbant assay (ELISA). The resulting mean serum concentration versus time profiles were then subjected to noncompartmental pharmacokinetic analyses. The following pharmacokinetic parameters were calculated: C₀ and Cₘₐₓ (extrapolated concentration at time zero and maximum serum concentration, respectively), Tₘₐₓ (time to achieve maximum concentration), t_{1/2 λz} (terminal half-life), AUC₀₋ₜ (area under the concentration versus time curve from time zero to the last measurable time point), AUC_{INF} (area under the concentration versus time curve extrapolated to infinity), C1 or C1/F (clearance or clearance divided by bioavailable fraction, respectively), V_{SS} or V_{Z}/F (steady state volume of distribution or volume of distribution divided by the bioavailable fraction, respectively), and F (bioavailable fraction). Results are summarized in Table 5.

TABLE 5

| Treatment | Parameter | Units | IV | IP | SC |
|---|---|---|---|---|---|
| IL-27RAm(mFc1) | Cₒ; Cₘₐₓ | μg/mL | 36.2 | 13.2 | 7.95 |
| | Tₘₐₓ | h | — | 1 | 3 |
| | AUC₀₋₂₄ | h*μg/mL | 157 | 98.2 | 56.5 |
| | AUC_{INF} | h*μg/mL | 162 | 102 | NE |
| | t_{1/2 λz} | h | 5.25 | 5.17 | NE |
| | Vₛₛ; V_{z}/F | mL | 3.05 | 7.33 | NE |
| | Cl; Cl/F | mL/h | 0.618 | 0.983 | NE |
| | F | — | — | 0.625 | 0.360 |
| IL-27RA-Fc5 | Cₒ; Cₘₐₓ | μg/mL | 122 | 35.2 | 21.0 |
| | Tₘₐₓ | h | — | 3 | 6 |
| | AUC₀₋₁₂₀ | h*μg/mL | 1400 | 1310 | 1130 |
| | AUC_{INF} | h*μg/mL | 1510 | 1380 | 1210 |
| | t_{1/2 λz} | h | 34.9 | 28.3 | 29.3 |
| | Vₛₛ; V_{z}/F | mL | 2.54 | 2.96 | 3.50 |
| | Cl; Cl/F | mL/h | 0.0664 | 0.0725 | 0.0828 |
| | F | — | — | 0.914 | 0.801 |

NE, not estimable due to an insufficient characterization of the terminal portion of the concentration versus time curve;
—, not applicable.

In summary, the human Fc5 fusion protein was found to have a much longer terminal half-life (t_{1/2 λz}) than the mouse Fc1 fusion. This difference in t_{1/2 λz} between the two proteins is due to a more rapid clearance of IL-27RAm(mFc1) compared to IL-27RA-Fc5.

### Example 8

A DNA construct encoding a fusion protein comprising the extracellular domain of mouse IL27RA with a C-terminal polyhistidine tag (CH6) was constructed via a 2-step PCR and homologous recombination using a DNA fragment encoding the extracellular domain of mouse IL27RA and pZMP40.

The PCR fragment encoding IL27RAm(CH6) was constructed to contain a 5′ overlap with the pZMP40 vector sequence in the 5′ non-translated region, the IL27RA extracellular domain coding region, the HIS tag coding sequence, and a 3′ overlap with the pZMP40 vector in the poliovirus internal ribosome entry site region. The first PCR amplification reaction used the 5′ oligonucleotide primer zc45069 (SEQ ID NO:25), the 3′ oligonucleotide primer zc46754 (SEQ ID NO:26), and a previously generated plasmid containing a mouse IL27RA cDNA as the template. The second PCR amplified the initial PCR product using the 5′ oligonucleotide primer zc20392 (SEQ ID NO:27), and the 3′ oligonucleotide primer zc46758 (SEQ ID NO:28).

The PCR amplification reaction conditions were one cycle of 95° C. for 2 minutes; then 35 cycles of 95° C. for 30 seconds, 55° C. for 30 seconds and 72° C. for 2 minutes; then one cycle of 72° C. for 10 minutes; followed by a 4° C. hold. The PCR reaction mixture was run on a 1.2% agarose gel, and the DNA fragment corresponding to the expected size was extracted from the gel using a commercially available gel extraction kit (QIAQUICK). The final PCR product was cloned using a commercially available kit (TOPO TA CLONING Kit; Invitrogen) according to the manufacturer's directions. Two μL of the cloning reaction mixture was used to transform chemically competent E. coli cells (ONE SHOT DH10B-T1), which were then plated onto LB AMP plates overnight. A colony that contained the correct insert sequence was grown up in LB AMP broth, and the plasmid was purified with a commercially available kit (QIAPREP Spin Miniprep kit). The plasmid clone was digested with EcoRI, and the IL27RAm(CH6) insert was excised and purified using a commercially available gel extraction kit (QIAQUICK).

The plasmid pZMP40 was digested with BglII prior to recombination in yeast with the gel-extracted IL27RAm(CH6) fragment. One hundred μL of competent yeast (S. cerevisiae) cells were combined with 10 μl (1 .micro.g) of the IL27RAm(CH6) insert DNA and 100 ng of BglII-digested pZMP40 vector, and the mix was transferred to a 0.2-cm electroporation cuvette. The yeast/DNA mixture was electropulsed using power supply settings of 0.75 kV (5 kV/cm), ∞ ohms, and 25 μF. Six hundred μL of 1.2 M sorbitol was added to the cuvette, and the yeast was plated in 300-μL aliquots onto two URA-D plates and incubated at 30° C. After about 72 hours, the Ura⁺ yeast transformants from a single plate were resuspended in 1 ml H₂O and spun briefly to pellet the yeast cells. The cell pellet was resuspended in 500 μL of lysis buffer (Example 3). The 500 μL of the lysis mixture was added to a microcentrifuge tube containing 300 μL acid-washed glass beads and 200 μL phenol-chloroform, vortexed for 2 minutes, and spun for 5 minutes in a microcentrifuge at maximum speed. Three hundred μL of the aqueous phase was transferred to a fresh tube, and the DNA was precipitated with 600 μL ethanol, followed by centrifugation for 10 minutes at maximum speed. The tube was decanted, and the DNA pellet was resuspended in 10 μL dH₂O.

Electrocompetent E. coli host cells were transformed with 5 μl of the yeast DNA preparation and plasmid DNA was isolated as disclosed in Example 3. The sequence of the insert DNA is shown in SEQ ID NO:29.

CHO DXB11 cells were transfected with BstB1-digested IL27RAm(CH6)/pZMP40 as disclosed in Example 3. The transfected cells were subjected to nutrient selection followed by step amplification to 200 nM methotrexate (MTX), then to 1 μM MTX. Tagged protein expression was confirmed by Western blot, and the CHO cell pool was scaled up for harvests for protein purification.

### Example 9

Binding experiments are carried out to compare the binding affinity of IL-27 antagonists for IL-27 receptor to the binding affinity of IL-27 itself. The comparator protein is ¹²⁵I-labeled, single-chain mouse IL-27 (designated “A1426F”). The protein comprises, from amino terminus to carboxyl terminus, a FLAG tag, mouse EBI3, a 17 amino acid linker, and mouse IL-27 p28.

For saturation binding studies, ¹²⁵I-labeled A1426F was titered from 100 nM to 195 pM in 1:2 serial dilutions with and without a constant amount of unlabeled A1426F at 1.micro.M. These preparations were incubated with BHK cells expressing both IL-27RA and gp130 (BHK-mIL-27R cells) for 4 hours on ice. The cells were then washed three times with ice-cold binding buffer (DMEM with 1 mg/mL BSA and 20 mM HEPES, pH≈7.5), then solublized with 1N NaOH. These lysates were then checked for bound A1426F by checking for radiation with a gamma counter. These three saturation binding studies yielded kD's of 0.9, 1.35, and 1.16 nM for an average kD of 1.14 nM.

For competition binding studies, ¹²⁵I-labeled A1426F (0.1 nM) was added to preparations of unlabeled A1426F, mouse IL-27 p28 with a C-terminal polyhistidine tag (A1406F), or an unrelated control protein titered from 50 nM to 7.6 pM in 1:3 serial dilutions. These preparations were incubated with BHK-mIL-27R cells for 4 hours on ice. The cells were then washed three times with ice-cold binding buffer, then solublized with 1N NaOH. These lysates were then checked for bound A1426F by checking for radiation with a gamma counter. A1426F was able to compete with ¹²⁵I-labeled A1426F for binding on BHK-mIL-27R cells. A1406F and control protein were unable to compete with labeled A1426F.

For a time course study, ¹²⁵I-labeled A1426F at 1 nM with and without a constant amount of unlabeled A1426F at 1 .micro.M was allowed to bind to BHK-mIL-27R cells on ice for different amounts of time (0.5, 1, 2, 4, or 6 hours). The cells were then washed three times with ice-cold binding buffer, then solubilized with 1N NaOH. These lysates were then checked for bound A1426F by checking for radiation with a gamma counter. Maximum binding was reached at 4 hours.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A method of treating aplastic anemia in a patient, comprising administering to a patient having aplastic anemia a therapeutically effective amount of an IL-27 antagonist in combination with a pharmaceutically acceptable vehicle.

2 (canceled)

3. The method of claim 1 wherein said antagonist is a soluble IL-27RA protein that binds to and reduces the activity of IL-27.

4. The method of claim 3 wherein said soluble IL-27RA protein is a disulfide linked dimer, wherein each chain of said dimer comprises an extracellular ligand-binding domain of an IL27RA joined to an immunoglobulin fragment comprising a heavy chain CH3 domain.

5. The method of claim 4 wherein each chain of said dimer further comprises an immunoglobulin hinge between the extracellular ligand binding domain and said CH3 domain.

6. The method of claim 4 wherein said immunoglobulin fragment is an immunoglobulin Fc fragment.

7. The method of claim 6 wherein said immunoglobulin Fc fragment is a wild-type Fc fragment.

8. The method of claim 6 wherein said immunoglobulin Fc fragment contains an amino acid substitution that reduces binding of said Fc fragment to Fc.gamma.RI, reduces complement fixation, or replaces a cysteine residue that normally forms a disulfide bond with an immunoglobulin light chain.

9. The method of claim 6 wherein said immunoglobulin Fc fragment consists of a sequence of amino acid residues selected from the group consisting of the sequences shown in FIGS. 1A-1C.

10 (canceled)

11. The method of claim 3 wherein said IL-27RA protein comprises amino acid residues 33 to 744 of SEQ ID NO:3.

12. The method of claim 1 wherein said antagonist comprises an antigen-binding site of an antibody and wherein said antagonist specifically binds to IL27RA, EBI3, IL-27 p28, or an EBI3/IL-27 p28 heterodimer.

13. The method of claim 12 wherein said antagonist is an antibody.

14. The method of claim 13 wherein said antibody is selected from the group consisting of a monoclonal antibody, a humanized monoclonal antibody, a monoclonal antibody that specifically binds to IL27RA, an Fv fragment, a single-chain Fv fragment, a Fab fragment, a Fab′ fragment, a F(ab′)₂ fragment, a diabody, a minibody, and a Pab-scFv fusion.

15-17. (canceled)

18. The method of claim 1 wherein said aplastic anemia is acquired aplastic anemia.

19. The method of claim 1 wherein said IL-27 antagonist is administered in combination with an IL-12 antagonist.

20. The method of claim 19 wherein said IL-12 antagonist is selected from the group consisting of anti-IL-12 antibodies, anti-IL-12 receptor antibodies, and soluble IL-12 receptors.

21. The method of claim 3 wherein the soluble IL-27RA protein is a disulfide linked dimer and wherein each chain of said dimer independently, from amino terminus to carboxyl terminus, is a Zcytor1 fragment with at least 80% sequence identity to SEQ ID NO:5 operably linked to an immunoglobulin fragment comprising a heavy chain CH3 domain.

22. The method of claim 21 wherein said Zcytor1 fragment of at least one of said two polypeptides consists of an amino acid sequence with at least 80% sequence identity to residues 33 to 514 of SEQ ID NO:5.

23. The method of claim 21 wherein said Zcytor1 fragment is at least 80% identical to residues 33 to 235 of SEQ ID NO:5.

24. The method of claim 21 wherein said Zcytor1 fragment has at least 80% sequence identity to SEQ ID NO:5 with the provisio that residue 41 is a Cys residue, residues 52-54 have a Cys-X-Trp residue sequence, residue 151 is a Trp residue, residue 207 is an Arg residue, and residues 217-221 are a WSXWS domain.

25-47. (canceled)

48. A method of increasing blood cell production in a patient having aplastic anemia, comprising administering to a patient a therapeutically effective amount of an IL-27 antagonist in combination with a pharmaceutically acceptable vehicle, wherein the IL-27RA antagonist is a disulfide linked dimer and wherein each chain of said dimer independently, from amino terminus to carboxyl terminus, is a Zcytor1 fragment with at least 80% sequence identity to SEQ ID NO:5 operably linked to an immunoglobulin fragment comprising a heavy chain CH3 domain.


SEQUENCE LISTING (34 sequences)

SEQ ID NO: 1 - protein, 232 aa, Homo sapiens
  VARIANT (3)...(3): Xaa = Lys or Arg
EPXSXDKTHTCPPCPAPEXXGXPSVFLFPPKPKDTLMISRTPEVTCVVVDVSHEDPEVKFNWYVDGVEVHNAKTKPREEQYXSTYRVVSVLTVLHQDWLNGKEYKCKVSNKALPXXIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 2 - DNA, 2232 bp, Artificial Sequence
  Engineered fusion protein
atgcggggaggcaggggcgcccctttctggctgtggccgctgcccaagctggcgctgctgcctctgttgtgggtgcttttccagcggacgcgtccccagggcagcgccgggccactgcagtgctacggagttggacccttgggcgacttgaactgctcgtgggagcctcttggggacctgggagccccctccgagttacacctccagagccaaaagtaccgttccaacaaaacccagactgtggcagtggcagccggacggagctgggtggccattcctcgggaacagctcaccatgtctgacaaactccttgtctggggcactaaggcaggccagcctctctggccccccgtcttcgtgaacctagaaacccaaatgaagccaaacgccccccggctgggccctgacgtggacttttccgaggatgaccccctggaggccactgtccattgggccccacctacatggccatctcataaagttctgatctgccagttccactaccgaagatgtcaggaggcggcctggaccctgctggaaccggagctgaagaccatacccctgacccctgttgagatccaagatttggagctagccactggctacaaagtgtatggccgctgccggatggagaaagaagaggatttgtggggcgagtggagccccattttgtccttccagacaccgccttctgctccaaaagatgtgtgggtatcagggaacctctgtgggacgcctggaggagaggaacctttgcttctatggaaggccccagggccctgtgtgcaggtgagctacaaagtctggttctgggttggaggtcgtgagctgagtccagaaggaattacctgctgctgctccctaattcccagtggggcggagtgggccagggtgtccgctgtcaacgccacaagctgggagcctctcaccaacctctctttggtctgcttggattcagcctctgccccccgtagcgtggcagtcagcagcatcgctgggagcacggagctactggtgacctggcaaccggggcctggggaaccactggagcatgtagtggactgggctcgagatggggaccccctggagaaactcaactgggtccggcttccccctgggaacctcagtgctctgttaccagggaatttcactgtcggggtcccctatcgaatcactgtgaccgcagtctctgcttcaggcttggcctctgcatcctccgtctgggggttcagggaggaattagcacccctagtggggccaacgctttggcgactccaagatgcccctccagggacccccgccatagcgtggggagaggtcccaaggcaccagcttcgaggccacctcacccactacaccttgtgtgcacagagtggaaccagcccctccgtctgcatgaatgtgagtggcaacacacagagtgtcaccctgcctgaccttccttggggtccctgtgagctgtgggtgacagcatctaccatcgctggacagggccctcctggtcccatcctccggcttcatctaccagataacaccgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 3 - protein, 744 aa, Artificial Sequence [sequence omitted]

SEQ ID NO: 4 - DNA, 2368 bp, Homo Sapiens
  CDS (23)...(1759)
ctcggggctcccgagggacgccatgcggggaggcaggggcgcccctttctggctgtggccgctgcccaagctggcgctgctgcctctgttgtgggtgcttttccagcggacgcgtccccagggcagcgccgggccactgcagtgctacggagttggacccttgggcgacttgaactgctcgtgggagcctcttggggacctgggagccccctccgagttacacctccagagccaaaagtaccgttccaacaaaacccagactgtggcagtggcagccggacggagctgggtggccattcctcgggaacagctcaccatgtctgacaaactccttgtctggggcactaaggcaggccagcctctctggccccccgtcttcgtgaacctagaaacccaaatgaagccaaacgccccccggctgggccctgacgtggacttttccgaggatgaccccctggaggccactgtccattgggccccacctacatggccatctcataaagttctgatctgccagttccactaccgaagatgtcaggaggcggcctggaccctgctggaaccggagctgaagaccatacccctgacccctgttgagatccaagatttggagctagccactggctacaaagtgtatggccgctgccggatggagaaagaagaggatttgtggggcgagtggagccccattttgtccttccagacaccgccttctgctccaaaagatgtgtgggtatcagggaacctctgtgggacgcctggaggagaggaacctttgcttctatggaaggccccagggccctgtgtgcaggtgagctacaaagtctggttctgggttggaggtcgtgagctgagtccagaaggaattacctgctgctgctccctaattcccagtggggcggagtgggccagggtgtccgctgtcaacgccacaagctgggagcctctcaccaacctctctttggtctgcttggattcagcctctgccccccgtagcgtggcagtcagcagcatcgctgggagcacggagctactggtgacctggcaaccggggcctggggaaccactggagcatgtagtggactgggctcgagatggggaccccctggagaaactcaactgggtccggcttccccctgggaacctcagtgctctgttaccagggaatttcactgtcggggtcccctatcgaatcactgtgaccgcagtctctgcttcaggcttggcctctgcatcctccgtctgggggttcagggaggaattagcacccctagtggggccaacgctttggcgactccaagatgcccctccagggacccccgccatagcgtggggagaggtcccaaggcaccagcttcgaggccacctcacccactacaccttgtgtgcacagagtggaaccagcccctccgtctgcatgaatgtgagtggcaacacacagagtgtcaccctgcctgaccttccttggggtccctgtgagctgtgggtgacagcatctaccatcgctggacagggccctcctggtcccatcctccggcttcatctaccagataacaccctgaggtggaaagttctgccgggcatcctattcttgtggggcttgttcctgttggggtgtggcctgagcctggccacctctggaaggtgctaccacctaaggcacaaagtactgccccgctgggtctgggagaaagttcctgatcctgccaacagcagttcaggccttctggggccccccaggccacaggttctggcctgaaccacacgtctggctgggggctgccagccaggctagagggatgctcatgcaggttgcaccccagtcctggattagccctcttgatggatgaagacactgaggactcagagaggctgagtcacttacctgaggacacccagccaggcagagctgggattgaaggacccctatagagaagggcttggcccccatggggaagacacggatggaaggtggagcaaaggaaaatacatgaaattgagagtggcagctgcctgccaaaatctgttccgctgtaacagaactgaatttggaccccagccagtggctcacgcctgtaatcccagcactttggcaggccaaggtggaaggatcacttagagctaggagtttgagaccagcctgggcaatatgcaagacccctcactacaaaaataaaacatcaaaaacaaaaacaattagctgggcatgatggcacacacctgtgtccgagccacttgggaggctgggtgggaggatcggttgagcccaggagttcgaagctgcagggacctctgattgcaccactgcactccaggctgggtaacagaatgagccttatctcaaaaataaacaaactaataaaaagta

SEQ ID NO: 5 - protein, 578 aa, Homo Sapiens
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGLLGPPRPQVLA

SEQ ID NO: 6 - DNA, 2589 bp, Mus musculus
  CDS (11)...(1882)
gctgggagccatgaaccggctccgggttgcacgcctcacgccgttggagcttctgctgtcgctgatgtcgctgctgctcgggacgcggccccacggcagtccaggcccactgcagtgctacagcgtcggtcccctgggaatcctgaactgctcctgggaacctttgggcgacctggagactccacctgtgctgtatcaccagagtcagaaataccatcccaatagagtctgggaggtgaaggtgccttccaaacagagttgggtgaccattccccgggaacagttcaccatggctgacaaactcctcatctgggggacacaaaagggacggcctctgtggtcctctgtctctgtgaacctggagacccaaatgaagccagacacacctcagatcttctctcaagtggatatttctgaggaagcaaccctggaggccactgtgcagtgggcgccgcccgtgtggccaccgcagaaagctctcacctgtcagttccggtacaaggaatgccaggctgaagcatggacccggctggagccccagctgaagacagatgggctgactcctgttgagatgcagaacctggaacctggcacctgctaccaggtgtctggccgctgccaggtggagaacggatatccatggggcgagtggagttcgcccctgtccttccagacgccattcttagatcctgaagatgtgtgggtatcggggaccgtctgtgaaacttctggcaaacgggcagccctgcttgtctggaaggacccaagaccttgtgtgcaggtgacttacacagtctggtttggggctggagatattactacaactcaagaagaggtcccgtgctgcaagtcccctgtccctgcatggatggagtgggctgtggtctctcctggcaacagcaccagctgggtgcctcccaccaacctgtctctggtgtgcttggctccagaatctgccccctgtgacgtgggagtgagcagtgctgatgggagcccagggataaaggtgacctggaaacaagggaccaggaaaccattggagtatgtggtggactgggctcaagatggtgacagcctggacaagctcaactggacccgtctcccccctggaaacctcagcacattgttaccaggggagttcaaaggaggggtaccctatcgaattacagtgactgcagtatactctggaggattagctgctgcaccctcagtttggggattcagagaggagttagtaccccttgctgggccagcagtttggcgacttccagatgaccccccagggacacctgttgtagcctggggagaagtaccaagacaccagctcagaggccaggctactcactacaccttctgcatacagagcagaggcctctccactgtctgcaggaacgtgagcagtcaaacccagactgccactctgcccaaccttcactcgggttccttcaagctgtgggtgacggtgtccaccgttgcaggacagggcccacctggtcccgacctttcacttcacctaccagataataggatcaggtggaaagctctgccctggtttctgtccctgtggggtttgcttctgatgggctgtggcctgagcctggccagtaccaggtgcctacaggccaggtgcttacactggcgacacaagttgcttccccagtggatctgggagagggttcctgatcctgccaacagcaattctgggcaaccttacatcaaggaggtgagcctgccccaaccgcccaaggacggacccatcctggaggtggaggaagtggagctacagcctgttgtggagtcccctaaagcctctgccccgatttactctgggtatgagaaacacttcctgcccacaccagaggagctgggccttctagtctgatctgcttacggctaggggctgtacccctatcttgggctagacgtttttgtatttttagatttttgagacaggatctcactatggctgacctggaacttgatataacaaccaggctggcctggaactcaccaagactcacctggttttgccttccaaggactgagaagaaatgagtgtgccgcctcccgcccaaccagcttttgctttccttgcctctgggttcttgggcatctgtttgttactgcagaagaatcagtgagctcacagcctcaaccccatcgttgttatttcctccttgtgtcacaggcttgctaggtagccaaggctggcctcgaacttgtgatcctccctgctgcagcatccccagagctgggattacaggtgtgcgtcacttcatcgagtcataacttttgattctagtaagaataactaccaggcaggctatgaaggtggtgactcgaaagacacattcaaggacctaaagtggttaagagcctgtgttttcttgcagtagaccaaagtttggttccctgcccttgcaaaggacacacgttcagtttccagcacccacagggcagctcagaatcacctgtaactccaggtccaaggaatccaatgccctcttctggcttctgtgagccccgcacacacatggttacttatgcaccgaaaaacacacgcataaaataaaaataaataaataaacc

SEQ ID NO: 7 - protein, 623 aa, Mus musculus
MNRLRVARLTPLELLLSLMSLLLGTRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIRWKALPWFLSLWGLLLMGCGLSLASTRCLQARCLHWRHKLLPQWIWERVPDPANSNSGQPYIKEVSLPQPPKDGPILEVEEVELQPVVESPKASAPIYSGYEKHFLPTPEELGLLV

SEQ ID NO: 8 - DNA, 18 bp, Artificial Sequence
  oligonucleotide primer zc10381
ccactacaccttgtgtgc

SEQ ID NO: 9 - DNA, 37 bp, Artificial Sequence
  oligonucleotide primer zc10390
tagtagcagatctgggctccctcagggtgttatctgg

SEQ ID NO: 10 - DNA, 20 bp, Artificial Sequence
  oligonucleotide primer zc10314
tcgtgattctctggtcggtg

SEQ ID NO: 11 - DNA, 20 bp, Artificial Sequence
  oligonucleotide primer zc10315
gtgattgctttggcggtgag

SEQ ID NO: 12 - DNA, 19 bp, Artificial Sequence
  oligonucleotide primer zc10382
gagttcaaaggaggggtac

SEQ ID NO: 13 - DNA, 40 bp, Artificial Sequence
  oligonucleotide primer zc10388
tagtagcagatctgggctccctgatcctattatctggtag

SEQ ID NO: 14 - DNA, 66 bp, Artificial Sequence
  oligonucleotide primer zc46250
ggtgtcctcgagaattcatataggccggccaccatgaaccggctccgggttgcacgcctcacgccg

SEQ ID NO: 15 - DNA, 43 bp, Artificial Sequence
  oligonucleotide primer zc47631
gcttgattgtgggccctctgggctccctgatcctattatctgg

SEQ ID NO: 16 - DNA, 22 bp, Artificial Sequence
  oligonucleotide primer zc24901
gagcccagagggcccacaatca

SEQ ID NO: 17 - DNA, 52 bp, Artificial Sequence
  oligonucleotide primer zc46896
gctgttttaaggcgcgcctctagatttatttacccggagtccgggagaagct

SEQ ID NO: 18 - DNA, 51 bp, Artificial Sequence
  oligonucleotide primer zc46759
tctggggtgggtacaaccccagagctgttttaaggcgcgcctctagattta

SEQ ID NO: 19 - DNA, 2226 bp, Artificial Sequence
  Engineered fusion protein
atgaaccggctccgggttgcacgcctcacgccgttggagcttctgctgtcgctgatgtcgctgctgctcgggacgcggccccacggcagtccaggcccactgcagtgctacagcgtcggtcccctgggaatcctgaactgctcctgggaacctttgggcgacctggagactccacctgtgctgtatcaccagagtcagaaataccatcccaatagagtctgggaggtgaaggtgccttccaaacagagttgggtgaccattccccgggaacagttcaccatggctgacaaactcctcatctgggggacacaaaagggacggcctctgtggtcctctgtctctgtgaacctggagacccaaatgaagccagacacacctcagatcttctctcaagtggatatttctgaggaagcaaccctggaggccactgtgcagtgggcgccgcccgtgtggccaccgcagaaagctctcacctgtcagttccggtacaaggaatgccaggctgaagcatggacccggctggagccccagctgaagacagatgggctgactcctgttgagatgcagaacctggaacctggcacctgctaccaggtgtctggccgctgccaggtggagaacggatatccatggggcgagtggagttcgcccctgtccttccagacgccattcttagatcctgaagatgtgtgggtatcggggaccgtctgtgaaacttctggcaaacgggcagccctgcttgtctggaaggacccaagaccttgtgtgcaggtgacttacacagtctggtttggggctggagatattactacaactcaagaagaggtcccgtgctgcaagtcccctgtccctgcatggatggagtgggctgtggtctctcctggcaacagcaccagctgggtgcctcccaccaacctgtctctggtgtgcttggctccagaatctgccccctgtgacgtgggagtgagcagtgctgatgggagcccagggataaaggtgacctggaaacaagggaccaggaaaccattggagtatgtggtggactgggctcaagatggtgacagcctggacaagctcaactggacccgtctcccccctggaaacctcagcacattgttaccaggggagttcaaaggaggggtaccctatcgaattacagtgactgcagtatactctggaggattagctgctgcaccctcagtttggggattcagagaggagttagtaccccttgctgggccagcagtttggcgacttccagatgaccccccagggacacctgttgtagcctggggagaagtaccaagacaccagctcagaggccaggctactcactacaccttctgcatacagagcagaggcctctccactgtctgcaggaacgtgagcagtcaaacccagactgccactctgcccaaccttcactcgggttccttcaagctgtgggtgacggtgtccaccgttgcaggacagggcccacctggtcccgacctttcacttcacctaccagataataggatcagggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaataa

SEQ ID NO: 20 - protein, 741 aa, Artificial Sequence
  Engineered fusion protein
MNRLRVARLTPLELLLSLMSLLLGTRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIREPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - DNA, 58 bp, Artificial Sequence
  Oligonucleotide primer zc53405
aggtgtccagctcgagaattcatataggccggccaccatgcggggaggcaggggcgcc

SEQ ID NO: 22 - DNA, 63 bp, Artificial Sequence
  Oligonucleotide primer zc51828
cggtgggcatgtgtgagttttgtctgaagatttgggctcggtgttatctggtagatgaagccg

SEQ ID NO: 23 - DNA, 63 bp, Artificial Sequence
  Oligonucleotide primer zc51827
cctggtcccatcctccggcttcatctaccagataacaccgagcccaaatcttcagacaaaact

SEQ ID NO: 24 - DNA, 62 bp, Artificial Sequence
  Oligonucleotide primer zc42508
ggggtgggtacaaccccagagctgttttaaggcgcgcctttatttacccggagacagggaga

SEQ ID NO: 25 - DNA, 60 bp, Artificial Sequence
  Oligonucleotide primer zc45069
aggtgtccagggaattcatataggccggccaccatgaaccggctccgggttgcacgcctc

SEQ ID NO: 26 - DNA, 45 bp, Artificial Sequence
  Oligonucleotide primer zc46754
tggtgatggtgatgtccaccagatcccctgatcctattatctggt

SEQ ID NO: 27 - DNA, 60 bp, Artificial Sequence
  Oligonucleotide primer zc20392
gacattgacatccactttgcctttctctccacaggtgtccagggaattcatataggccgg

SEQ ID NO: 28 - DNA, 59 bp, Artificial Sequence
  Oligonucleotide primer zc46758
aaccccagagctgttttaaggcgcgcctctagatttagtgatggtgatggtgatgtcca

SEQ ID NO: 29 - DNA, 1557 bp, Artificial Sequence
  Engineered fusion protein
atgaaccggctccgggttgcacgcctcacgccgttggagcttctgctgtcgctgatgtcgctgctgctcgggacgcggccccacggcagtccaggcccactgcagtgctacagcgtcggtcccctgggaatcctgaactgctcctgggaacctttgggcgacctggagactccacctgtgctgtatcaccagagtcagaaataccatcccaatagagtctgggaggtgaaggtgccttccaaacagagttgggtgaccattccccgggaacagttcaccatggctgacaaactcctcatctgggggacacaaaagggacggcctctgtggtcctctgtctctgtgaacctggagacccaaatgaagccagacacacctcagatcttctctcaagtggatatttctgaggaagcaaccctggaggccactgtgcagtgggcgccgcccgtgtggccaccgcagaaagctctcacctgtcagttccggtacaaggaatgccaggctgaagcatggacccggctggagccccagctgaagacagatgggctgactcctgttgagatgcagaacctggaacctggcacctgctaccaggtgtctggccgctgccaggtggagaacggatatccatggggcgagtggagttcgcccctgtccttccagacgccattcttagatcctgaagatgtgtgggtatcggggaccgtctgtgaaacttctggcaaacgggcagccctgcttgtctggaaggacccaagaccttgtgtgcaggtgacttacacagtctggtttggggctggagatattactacaactcaagaagaggtcccgtgctgcaagtcccctgtccctgcatggatggagtgggctgtggtctctcctggcaacagcaccagctgggtgcctcccaccaacctgtctctggtgtgcttggctccagaatctgccccctgtgacgtgggagtgagcagtgctgatgggagcccagggataaaggtgacctggaaacaagggaccaggaaaccattggagtatgtggtggactgggctcaagatggtgacagcctggacaagctcaactggacccgtctcccccctggaaacctcagcacattgttaccaggggagttcaaaggaggggtaccctatcgaattacagtgactgcagtatactctggaggattagctgctgcaccctcagtttggggattcagagaggagttagtaccccttgctgggccagcagtttggcgacttccagatgaccccccagggacacctgttgtagcctggggagaagtaccaagacaccagctcagaggccaggctactcactacaccttctgcatacagagcagaggcctctccactgtctgcaggaacgtgagcagtcaaacccagactgccactctgcccaaccttcactcgggttccttcaagctgtgggtgacggtgtccaccgttgcaggacagggcccacctggtcccgacctttcacttcacctaccagataataggatcaggggatctggtggacatcaccatcaccatcactaa

SEQ ID NO: 30 - protein, 518 aa, Artificial Sequence
  Engineered fusion protein
MNRLRVARLTPLELLLSLMSLLLGTRPHGSPGPLQCYSVGPLGILNCSWEPLGDLETPPVLYHQSQKYHPNRVWEVKVPSKQSWVTIPREQFTMADKLLIWGTQKGRPLWSSVSVNLETQMKPDTPQIFSQVDISEEATLEATVQWAPPVWPPQKALTCQFRYKECQAEAWTRLEPQLKTDGLTPVEMQNLEPGTCYQVSGRCQVENGYPWGEWSSPLSFQTPFLDPEDVWVSGTVCETSGKRAALLVWKDPRPCVQVTYTVWFGAGDITTTQEEVPCCKSPVPAWMEWAVVSPGNSTSWVPPTNLSLVCLAPESAPCDVGVSSADGSPGIKVTWKQGTRKPLEYVVDWAQDGDSLDKLNWTRLPPGNLSTLLPGEFKGGVPYRITVTAVYSGGLAAAPSVWGFREELVPLAGPAVWRLPDDPPGTPVVAWGEVPRHQLRGQATHYTFCIQSRGLSTVCRNVSSQTQTATLPNLHSGSFKLWVTVSTVAGQGPPGPDLSLHLPDNRIRGSGGHHHHHH

SEQ ID NO: 31 - DNA, 101 bp, Artificial Sequence
  Oligonucleotide primer zc52311
tgggtacaaccccagagctgttttaaggcgcgcctctagattattttttagtgatggtgatggtgatgtccaccagatcccagggtgttatctggtagatg

SEQ ID NO: 32 - DNA, 1569 bp, Artificial Sequence
  Engineered fusion protein
atgcggggaggcaggggcgcccctttctggctgtggccgctgcccaagctggcgctgctgcctctgttgtgggtgcttttccagcggacgcgtccccagggcagcgccgggccactgcagtgctacggagttggacccttgggcgacttgaactgctcgtgggagcctcttggggacctgggagccccctccgagttacacctccagagccaaaagtaccgttccaacaaaacccagactgtggcagtggcagccggacggagctgggtggccattcctcgggaacagctcaccatgtctgacaaactccttgtctggggcactaaggcaggccagcctctctggccccccgtcttcgtgaacctagaaacccaaatgaagccaaacgccccccggctgggccctgacgtggacttttccgaggatgaccccctggaggccactgtccattgggccccacctacatggccatctcataaagttctgatctgccagttccactaccgaagatgtcaggaggcggcctggaccctgctggaaccggagctgaagaccatacccctgacccctgttgagatccaagatttggagctagccactggctacaaagtgtatggccgctgccggatggagaaagaagaggatttgtggggcgagtggagccccattttgtccttccagacaccgccttctgctccaaaagatgtgtgggtatcagggaacctctgtgggacgcctggaggagaggaacctttgcttctatggaaggccccagggccctgtgtgcaggtgagctacaaagtctggttctgggttggaggtcgtgagctgagtccagaaggaattacctgctgctgctccctaattcccagtggggcggagtgggccagggtgtccgctgtcaacgccacaagctgggagcctctcaccaacctctctttggtctgcttggattcagcctctgccccccgtagcgtggcagtcagcagcatcgctgggagcacggagctactggtgacctggcaaccggggcctggggaaccactggagcatgtagtggactgggctcgagatggggaccccctggagaaactcaactgggtccggcttccccctgggaacctcagtgctctgttaccagggaatttcactgtcggggtcccctatcgaatcactgtgaccgcagtctctgcttcaggcttggcctctgcatcctccgtctgggggttcagggaggaattagcacccctagtggggccaacgctttggcgactccaagatgcccctccagggacccccgccatagcgtggggagaggtcccaaggcaccagcttcgaggccacctcacccactacaccttgtgtgcacagagtggaaccagcccctccgtctgcatgaatgtgagtggcaacacacagagtgtcaccctgcctgaccttccttggggtccctgtgagctgtgggtgacagcatctaccatcgctggacagggccctcctggtcccatcctccggcttcatctaccagataacaccctgggatctggtggacatcaccatcaccatcac

SEQ ID NO: 33 - protein, 523 aa, Artificial Sequence
  Engineered fusion protein
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLGSGGHHHHHH

SEQ ID NO: 34 - protein, 636 aa, Homo sapiens
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA